(19) United States
(12) Patent Application Publication
(10) Pub. No.: US 2015/0090264 A1
(43) Pub. Date: Apr. 2, 2015

(54) METHODS AND SYSTEMS FOR PROPORTIONAL ASSIST VENTILATION

(71) Applicant: Covidien LP, Boulder, CO (US)

(72) Inventor: Nancy F. Dong, Carlsbad, CA (US)

(73) Assignee: Covidien LP, Boulder, CO (US)

(21) Appl. No.: 14/044,431

(22) Filed: Oct. 2, 2013

(51) Int. Cl. A61M 16/00; A61M 16/08; A61B 5/00

(52) U.S. Cl. CPC A61M 16/0051; A61M 16/0069; A61M 16/0003; A61B 5/4836; A61M 16/0883; A61M 2016/0039; A61B 5/087
USPC 128/204.23

(57) ABSTRACT

The systems and methods include providing a negative proportional assist breath type, a time adjusted negative proportional assist breath type, or a time adjusted proportional assist breath type during ventilation of a patient with a ventilator.

### INTRODUCTION

Medical ventilator systems have long been used to provide ventilatory and supplemental oxygen support to patients. These ventilators typically comprise a source of pressurized gas, such air or oxygen, which is fluidly connected to the patient through a conduit or tubing. As each patient may require a different ventilation strategy, modern ventilators can be customized for the particular needs of an individual patient. For example, several different ventilator modes or settings have been created to provide better ventilation for patients in various different scenarios.

### Proportional Assist Ventilation

This disclosure describes systems and methods for providing a negative proportional assist (NPA) breath type, a time adjusted negative proportional assist (TANPA) breath type, or a time adjusted proportional assist (TAPA) breath type during ventilation of a patient. In part, the disclosure describes a novel breath type that delivers a target inspiration pressure calculated based on a set pressure support level, a time delay caused by a control system, and an estimated patient effort estimated from the last computational cycle. In part, the disclosure describes a novel breath type that delivers a target inspiration pressure calculated based on a set pressure support level and an estimated patient effort estimated from the last computational cycle utilizing an injected inverse model principle. In part, the disclosure describes a novel breath type that delivers a target inspiration pressure calculated based on a set pressure support level, a time delay caused by a control system, and an estimated patient effort estimated from the last computational cycle utilizing an injected inverse model principle.

In part, this disclosure describes a method for ventilating a patient with a ventilator. The method includes:

delivering an initial inspiration pressure to a patient in a first computational cycle;

retrieving a support setting;

monitoring inspiration flow during the first computation cycle;

estimating a first patient effort utilizing an inverse model based at least on the inspiration flow monitored during the first computational cycle;

calculating a first target inspiration pressure based at least on the first estimated patient effort from the first computational cycle and the support setting; and

delivering the first target inspiration pressure to the patient in a second computational cycle.

Yet another aspect of this disclosure describes a method for ventilating a patient with a ventilator. This method includes:

delivering an initial inspiration pressure to a patient in a first computational cycle;

retrieving a support setting;

monitoring inspiration flow during the first computational cycle;

estimating a first patient effort utilizing at least the inspiration flow monitored during the first computational cycle;

calculating a first target inspiration pressure based at least on the first estimated patient effort from the first computational cycle, the support setting, and a time delay caused by a control system of the ventilator; and

delivering the first target inspiration pressure to the patient in a second computational cycle.

An additional aspect of this disclosure describes a ventilator system. This ventilator system includes a pressure generating system, a ventilation tubing system, one or more sensors, an inverse model (IM) effort module, and a negative proportional assist module. The pressure generating system generates a flow of breathing gas. The ventilation tubing system includes a patient interface for connecting the pressure generating system to a patient. The one or more sensors operatively couple to at least one of the pressure generating system, the patient, and the ventilation tubing system. The one or more sensors generate output indicative of at least an inspiration flow. The inverse model effort module calculates an estimated patient effort for each computational cycle utilizing an inverse model based on the output indicative of at least the inspiration flow from a last computational cycle. The negative proportional assist module receives a support setting, receives an estimated patient effort from the IM effort module for each computational cycle, calculates a target inspiration pressure based at least on the received support setting and the estimated patient effort received from the IM effort module for the last computational cycle, and sends instructions to the pressure generating system to deliver the calculated target inspiration pressure in a next computational cycle to the patient during a negative proportional assist (NPA) breath type. The instructions sent by the IM effort module and the NPA module provide closed-loop ventilation that is a negative feedback system.

Another aspect of this disclosure describes a ventilator system. This ventilator system includes a pressure generating system, a ventilation tubing system, one or more sensors, an effort module, and a proportional assist module. The pressure generating system generates a flow of breathing gas. The ventilation tubing system includes a patient interface for connecting the pressure generating system to a patient. The one or more sensors are operatively coupled to at least one of the pressure generating system, the patient, and the ventilation tubing system. The one or more sensors generate output indicative of at least an inspiration flow. The effort module calculates an estimated patient effort for each computational cycle based on the output indicative of at least the inspiration flow from a last computational cycle. The proportional assist module receives a support setting, receives the estimated patient effort for each computational cycle from the effort module, calculates a target pressure based on the received support setting, the estimated patient effort from the last computational cycle, and a time delay caused by a control system of the ventilator system, and sends instructions to the pressure generating system to deliver the calculated target inspiration pressure in a next computational cycle to the patient.

A further aspect of this disclosure describes a non-transitory computer-readable medium having computer-executable instructions executed by a processor of a controller. The controller including:

instructions to estimate a first patient effort utilizing an inverse model based at least on a monitored inspiration flow during a last computational cycle to a patient;

instructions to receive a support setting,

instructions to receive an estimated patient effort for the last computational cycle;

instructions to calculate a target inspiration pressure based at least on the estimated patient effort from the last computational cycle and the received support setting; and

instructions to send commands to a pressure generation system to deliver the target inspiration pressure delivered to the patient in a next computational cycle.

The executed instructions from the controller provide closed-loop ventilation that is a negative feedback system.

Yet another aspect of this disclosure describes a non-transitory computer-readable medium having computer-executable instructions executed by a processor of a controller. The controller including:

instructions to estimate a first patient effort based at least on a monitored inspiration flow during a last computational cycle to a patient;

instructions to receive a support setting,

instructions to receive an estimated patient effort for the last computational cycle;

instructions to calculate a target inspiration pressure based at least on the estimated patient effort from the last computational cycle and the received support setting, and a time delay caused by a control system; and

instructions to send commands to a pressure generation system to deliver the target inspiration pressure delivered to the patient in a next computational cycle.

Another aspect of this disclosure describes a method for ventilating a patient with a ventilator. The method including:

retrieving a support setting;

monitoring inspiration flow during a first computational cycle;

estimating a first patient effort utilizing an inverse model based at least on the monitored inspiration flow during the first computational cycle;

calculating a first target inspiration pressure based at least on the estimated patient effort from the first computational cycle and the support setting; and

delivering the first target inspiration pressure to the patient in a second computational cycle.

In part, this disclosure describes a method for ventilating a patient with a ventilator. The method includes:

retrieving a support setting;

monitoring inspiration flow during a first computational cycle;

estimating a first patient effort utilizing at least the monitored inspiration flow during the first computational cycle;

calculating a first target inspiration pressure based at least on the estimated patient effort from the first computational cycle, the support setting, and a time delay caused by a control system of the ventilator; and

delivering the first target inspiration pressure to the patient in a second computational cycle.

In part, this disclosure describes non-transitory computer-readable medium having computer-executable instructions executed by a processor of a controller. The controller includes an inverse model effort module and a negative proportional assist module. The inverse model effort module estimates a first patient effort utilizing an inverse model based at least on a monitored inspiration flow during a last computational cycle to a patient. The NPA module receives a support setting, receives an estimated patient effort for the last computational cycle from the IM effort module; calculates a target inspiration pressure based at least on the estimated patient effort from the last computational cycle and the received support setting; and sends commands to a pressure generation system to deliver the target inspiration pressure delivered to the patient in a next computational cycle. The executed instructions from the controller provide for closed-loop ventilation that is a negative feedback system.

This disclosure also describes non-transitory computer-readable medium having computer-executable instructions executed by a processor of a controller. The controller includes an effort module and a time adjusted proportional assist module. The effort module estimates a first patient effort based at least on a monitored inspiration flow during a last computational cycle to a patient. The time adjusted proportional assist module receives a support setting, receives an estimated patient effort for the last computational cycle from the effort module, calculates a target inspiration pressure based at least on the estimated patient effort from the last computational cycle and the received support setting, and a time delay caused by a control system, and sends commands to a pressure generation system to deliver the target inspiration pressure delivered to the patient in a next computational cycle.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments of systems and methods described below and are not meant to limit the scope of the invention in any manner, which scope shall be based on the claims appended hereto.

FIG. 1 illustrates an embodiment of a ventilator.

FIG. 2 illustrates an embodiment of a method for ventilating a patient with a ventilator utilizing a NPA breath type.

FIG. 3 illustrates an embodiment of a method for ventilating a patient with a ventilator utilizing a TANPA breath type.

FIG. 4 illustrates an embodiment of a method for ventilating a patient with a ventilator utilizing a TAPA breath type.

FIG. 5 illustrates an embodiment of a NPA breath type scheme based on an injected inverse model principle.

FIG. 6 illustrate a stability margin comparison of a negative proportional assist breath type and a proportional assist breath type using Nyquist plots of simulated under-estimated respiratory parameters.

FIG. 7 illustrate a stability margin comparison of a negative proportional assist breath type and a proportional assist breath type using Nyquist plots of over-estimated simulated respiratory parameters.

FIG. 8 illustrates an embodiment of a ventilator control system scheme.

FIG. 9 illustrates an embodiment of a ventilator control system scheme.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. A person of skill in the art will understand that the technology described in the context of a medical ventilator for human patients could be adapted for use with other systems such as ventilators for non-human patients and general gas transport systems.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

While operating a ventilator, it is desirable to control the percentage of oxygen in the gas supplied by the ventilator to the patient. Further, as each patient may require a different ventilation strategy, modern ventilators can be customized for the particular needs of an individual patient. For example, several different ventilator breath types have been created to provide better ventilation for patients in various different scenarios.

Effort-based breath types, such as proportional assist (PA) ventilation, dynamically determine the amount of ventilatory support to deliver based on a continuous estimation/calculation of patient effort and respiratory characteristics. The resulting dynamically generated profile is computed in real- or quasi-real-time and used by the ventilator as a set of points for control of applicable parameters.

Initiation and execution of an effort-based breath, such as PA, has two operation prerequisites: (1) detection of an inspiratory trigger; and (2) detection and measurement of an appreciable amount of patient respiratory effort to constitute a sufficient reference above a ventilator's control signal error deadband. Advanced, sophisticated triggering technologies detect initiation of inspiratory efforts. In ventilation design, patient effort may be represented by the estimated inspiratory muscle pressure and is calculated based on measured patient inspiration flow. Patient effort is utilized to calculate a target inspiration pressure for the inspiration. The target inspiration pressure as used herein is calculated on an on-going basis based on estimated patient effort according to the equation of motion and a support setting. In other words, the target inspiration pressure is the amount of pressure delivered by the ventilator to the patient.

A PA breath type refers to a type of ventilation in which the ventilator acts as an inspiratory amplifier that provides pressure support based on the patient's effort. The degree of amplification (the “support setting”) during a PA breath type is set or selected by an operator, for example as a percentage based on the patient's effort. In one implementation of a PA breath type, the ventilator may continuously monitor the patient's instantaneous inspiratory flow and instantaneous net lung volume, which are indicators of the patient's inspiratory effort. These signals, together with ongoing estimates of the patient's lung compliance and lung/airway resistance and the Equation of Motion (Target Pressure(t)=Eₚ∫Qₚdt+QₚRₚ−Patient Effort(t)), allow the ventilator to estimate/calculate a patient effort. The patient effort is calculated utilizing a positive feedback system. The target inspiration pressure is derived from the estimated patient effort to provide the support that assists the patient's inspiratory muscles to the degree selected by the operator as the support setting. Qₚ is the instantaneous flow inhaled by the patient, and Eₚ and Rₚ are the patient's respiratory elastance and resistance, respectively. E_{P} accounts for the patient lung elastance and the patient's chest wall elastance. Similarly, R_{P} accounts for the patient's chest wall resistance and the patient's lung resistance. In this equation one common measure of the patient effort is inspiratory muscle pressure (also referred to as Pₘᵤₛ). The support setting (β) input by the operator divides the total work of breathing calculated between the patient and the ventilator as shown in the equations below:

Pₘᵤₛ(t)=(1.0−β)[Eₚ∫Qₚdt+QₚRₚ] and 1)

Target Airway Pressure(t)=β[Eₚ∫Qₚdt+QₚRₚ] 2)

Pₘᵤₛ is the amount of pressure provided by the patient's muscles, Target inspiration pressure (also referred to herein as “Pᵥₑₙₜ”) is the amount of pressure provided by the ventilator, t stands for the time in a continuous domain, the total pressure delivered to the patient is [Eₚ∫Qₚdt+QₚRₚ] or the sum of contributions by the patient and ventilator, and β is the support setting (i.e., percentage or ratio of total support to be contributed by the ventilator) input or selected by the operator.

In theory, with a PA breath type, the target pressure is proportional to the patient effort (i.e. the patient's inspiratory muscle pressure (Pₘᵤₛ)). During a PA breath type, the ventilator assumes that there is automatic synchrony between the end of the patient's effort and of the ventilator cycling the inspiratory flow off. In practice, however, there are three main drawbacks to the PA breath type: (1) The closed-loop system in the PA breath type is a positive feedback system, which may easily lead the system away from stability; (2) A “Run-away” phenomenon commonly exists in the PA breath type when the pressure delivered by the ventilator is more than the pressure that is needed by the patient (also known as excessive assist); and (3) Asynchrony may exist between the patient and ventilator because the PA breath type does not estimate the patient inspiratory effort directly.

Accordingly, the current disclosure describes a PA breath type that utilizes a negative feedback system based on an Inverse model principle to estimate patient effort and is referred to herein as a Negative Pressure Assist (NPA) breath type. The negative feedback system of the NPA breath type provides a more stable and more accurate estimate of patient effort and prevents or reduces the likelihood of a run-away when compared to the conventional PA breath type. This more stable estimated patient effort is then used to generate the target pressure of the ventilator. Because the estimate of patient effort is more accurate during the NPA breath type, so too, is the ventilator support, improving the synchrony between the ventilator and the patient when compared to the conventional PA breath type. The respiratory parameters (including resistance and elastance) are identified by using a recursive least square (RLS) based adaptive algorithm.

Additionally, as discussed above, the PA breath type assumes that there is automatic synchrony between the end of the patient's effort and the ventilator cycling of the inspiratory flow off. In practice, however, expiratory asynchrony often occurs. Expiratory asynchrony is a phenomenon that happens when the ventilator's transition from inhalation phase to exhalation phase occurs before or after the end of the patient's inspiratory effort. Expiratory asynchrony causes discomfort to the patient and negatively affects patient's inspiratory/expiratory patient effort and ventilator triggering response. One contributor of expiratory asynchrony is the control system time delay in medical ventilators, i.e. the time lag between the input (e.g. the measured patient airway pressure or flow) and the output (e.g. the pressure or flow delivered by the ventilator). The control system as used herein refers to any portions of the ventilator that are utilized to control the gas delivery of the ventilator, such as an analog or digital controller, valve, inspiratory module, expiratory module, flow sensor, pressure sensor, and/or software. It was discovered that during a PA breath type, the time delay may be larger than in other breath types because the target for the PA breath type control system is pressure, which is a function of the combination of patient's flow, respiratory mechanics, and patient's spontaneous effort; and hence a more complex and time-consuming control logic is needed when compared to other breath types. As a result, expiratory asynchrony due to the control system time delay during the PA breath type is more significant than that in other breath types. The termination of ventilator flow lags to the completion of the patient's inspiratory flow by as much as the control system time delay.

Accordingly, the current disclosure describes a PA breath type that utilizes a dynamic assist ratio (DAR) and is referred to herein as a Time Adjusted Pressure Assist (TAPA) breath type. The DAR provides a TAPA breath type that effectively minimizes or eliminates expiratory asynchrony caused by a control system time delay when compared to the conventional PA breath type. Because the DAR adjusts for the control system time delay, the TAPA breath type improves the synchrony between the ventilator and the patient when compared to the conventional PA breath type. In some embodiments, the current disclosure describes a PA breath type that utilizes both the negative feedback system and the DAR and is referred to herein as a Time Adjusted Negative Pressure Assist (TANPA) breath type.

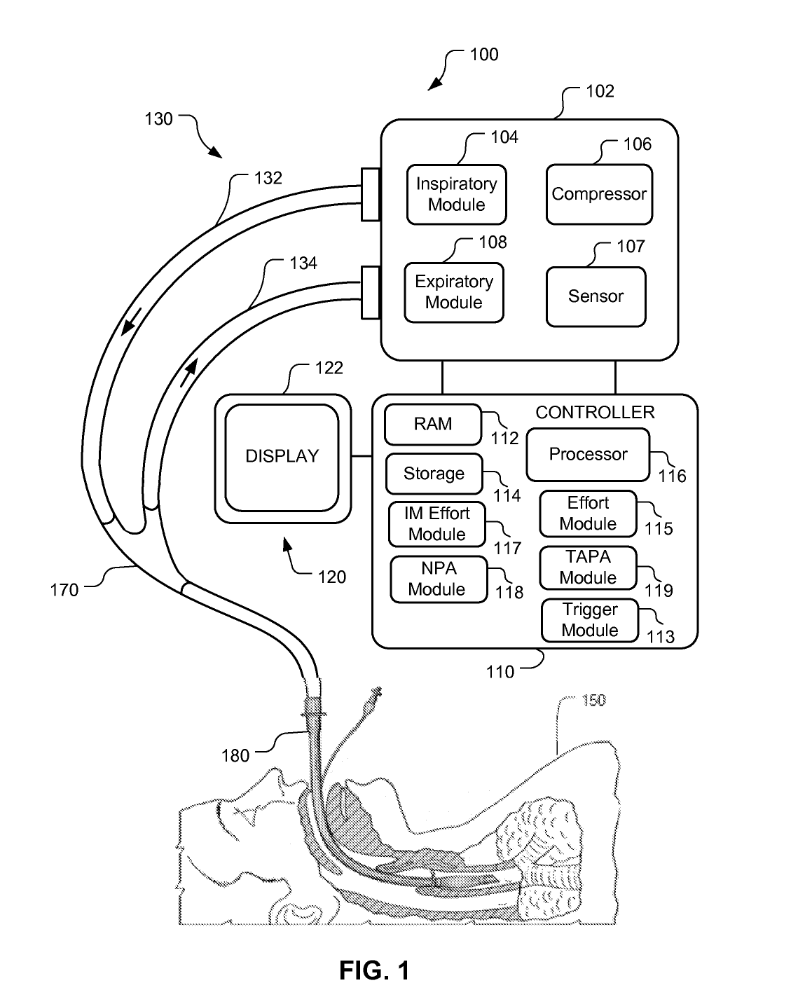

FIG. 1 is a diagram illustrating an embodiment of an exemplary ventilator 100 connected to a human patient 150. Ventilator 100 includes a pneumatic system 102 (also referred to as a pressure generating system 102) for circulating breathing gases to and from the patient 150 via the ventilation tubing system 130, which couples the patient 150 to the pneumatic system 102 via an invasive (e.g., endotracheal tube, as shown) or a non-invasive (e.g., nasal mask) patient interface 180.

Ventilation tubing system 130 (or patient circuit 130) may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, pneumatic system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 and the expiratory module 108 to provide a gas source for ventilatory support via inspiratory limb 132.

The inspiratory module 104 is configured to deliver gases to the patient 150 according to prescribed ventilatory settings. Specifically, inspiratory module 104 is associated with and/or controls one or more inspiratory valves for delivering gases to the patient 150 from a compressor 106 or another gas source.

The expiratory module 108 is configured to release gases from the patient's lungs according to prescribed ventilatory settings. Specifically, expiratory module 108 is associated with and/or controls one or more expiratory valves for releasing gases from the patient 150.

In some embodiments, pneumatic system 102, inspiratory module 104 and/or expiratory module 108 is/are configured to provide ventilation according to various breath types, e.g., via volume-control, pressure-control, pressure assist (PA), negative pressure assist (NPA), Time Adjusted Pressure Assist (TAPA), Time Adjusted Negative Pressure Assist (TANPA), or via any other suitable breath types.

The ventilator 100 may also include one or more sensors 107 communicatively coupled to ventilator 100. The sensors 107 may be located in the pneumatic system 102, ventilation tubing system 130, and/or on the patient 150. The embodiment of FIG. 1 illustrates a sensor 107 in pneumatic system 102.

Sensors 107 may communicate with various components of ventilator 100, e.g., pneumatic system 102, other sensors 107, processor 116, controller 110, trigger module 113, IM effort module 117, effort module 115, NPA module 118, TAPA module 119, and any other suitable components and/or modules. In one embodiment, sensors 107 generate output and send this output to pneumatic system 102, other sensors 107, processor 116, controller 110, trigger module 113, IM effort module 117, effort module 115, NPA module 118, TAPA module 119 and any other suitable components and/or modules. Sensors 107 may employ any suitable sensory or derivative technique for monitoring one or more patient parameters or ventilator parameters associated with the ventilation of a patient 150.

As used herein, patient parameters are any parameters determined based on measurements taken of the patient 150, such as heart rate, respiration rate, a blood oxygen level (SpO₂), inspiratory lung flow, airway pressure, and etc. As used herein, ventilator parameters are parameters that are determined by the ventilator 100 and/or are input into the ventilator 100 by an operator, such as a breath type, desired patient effort, support setting, and etc. Some parameters may be either or both ventilator and patient parameters depending upon whether or not they are input into the ventilator 100 by an operator or determined by the ventilator 100.

Sensors 107 may detect changes in patient parameters indicative of patient triggering, for example. Sensors 107 may be placed in any suitable location, e.g., within the ventilatory circuitry or other devices communicatively coupled to the ventilator 100. Further, sensors 107 may be placed in any suitable internal location, such as, within the ventilatory circuitry or within components or modules of ventilator 100. For example, sensors 107 may be coupled to the inspiratory and/or expiratory modules for detecting changes in, for example, circuit pressure and/or flow. In other examples, sensors 107 may be affixed to the ventilatory tubing or may be embedded in the tubing itself. According to some embodiments, sensors 107 may be provided at or near the lungs (or diaphragm) for detecting a pressure in the lungs. Additionally or alternatively, sensors 107 may be affixed or embedded in or near a wye-fitting 170 and/or patient interface 180. Indeed, any sensory device useful for monitoring changes in measurable parameters during ventilatory treatment may be employed in accordance with embodiments described herein.

As should be appreciated, with reference to the Equation of Motion, ventilatory parameters are highly interrelated and, according to embodiments, may be either directly or indirectly monitored. That is, parameters may be directly monitored by one or more sensors 107, as described above, or may be indirectly monitored or estimated/calculated using a model, such as a model derived from the Equation of Motion (e.g., Target Airway Pressure(t)=Eₚ∫Qₚ dt+QₚRₚ−Patient Effort(t)).

For example, sensor(s) 107 may include a flow sensor and/or a pressure sensor. These sensors 107 generate output showing the flow and/or the pressure of breathing gas delivered to the patient 150, exhaled by the patient 150, at the circuit wye, delivered by the ventilator 100, and/or within the ventilation tubing system 130. In some embodiments, a differential pressure transducer or sensor is utilized to calculate flow. Accordingly, a flow sensor as used herein includes a pressure sensor and a pressure sensor as used herein includes a flow sensor. In some embodiments, net volume, tidal volume, inspiratory volume, and/or an expiratory volume of the patient 150 are determined based on the sensor output from the flow sensor and/or pressure sensor.

The pneumatic system 102 may include a variety of other components, including mixing modules, valves, tubing, accumulators, filters, etc. Controller 110 is operatively coupled with pneumatic system 102, signal measurement and acquisition systems, sensor 107, display 122, and an operator interface 120 that may enable an operator to interact with the ventilator 100 (e.g., change ventilator settings, select operational modes, view monitored parameters, etc.).

In one embodiment, the operator interface 120 of the ventilator 100 includes a display 122 communicatively coupled to ventilator 100. Display 122 provides various input screens, for receiving clinician input, and various display screens, for presenting useful information to the clinician. In one embodiment, the display 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilator 100 may be provided, for instance by a wheel, keyboard, mouse, or other suitable interactive device. Thus, operator interface 120 may accept commands and input through display 122. Display 122 may also provide useful information in the form of various ventilatory data regarding the physical condition of a patient 150. The useful information may be derived by the ventilator 100, based on data collected by a processor 116 or controller 110, and the useful information may be displayed to the clinician in the form of graphs, wave representations, pie graphs, text, or other suitable forms of graphic display. For example, patient data may be displayed on the GUI and/or display 122. Additionally or alternatively, patient data may be communicated to a remote monitoring system or display coupled via any suitable way to the ventilator 100. In one embodiment, the display 122 may display one or more of the breath type, the estimated patient effort, the calculated target pressure, the total pressure delivered, the monitored inspiration pressure, the monitored net lung volume, an initial inspiratory pressure, a list of delivered target inspiration pressures for a predetermined number of computational cycles, a list of estimated patient efforts from a predetermined number of computational cycles, a graph of the list of the delivered target inspiration pressure and/or the estimated patient efforts for a predetermined number of computational cycle, an average delivered target inspiration pressure for a predetermined number of computational cycles, an averaged estimated patient effort from a predetermined number of computational cycles, a graph of the list of averaged delivered target inspiration pressures and/or averaged estimated patient efforts for a predetermined number of computational cycle for a set time period, the support setting, a volume-assist setting, a flow-assist setting, and/or a time delay caused by the control system.

Controller 110 may include memory 112, one or more processors 116, storage 114, and/or other components of the type commonly found in command and control computing devices. Controller 110 may further include an IM effort module 117, trigger module 113, an effort module 115, a NPA module 118, and/or a TAPA module 119 as illustrated in FIG. 1. The controller 110 is configured to deliver gases to the patient 150 according to prescribed or selected breath types. In alternative embodiments, the IM effort module 117, effort module 115, trigger module 113, the NPA module 118, and the TAPA module 119 may be located in other components of the ventilator 100, such as the pressure generating system 102 (also known as the pneumatic system 102).

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

The pneumatic system 102 receives a breath type, such as a PA, NPA, TAPA, or TANPA breath type, from the controller 110. The controller 110 receives the breath type from operator input or from a predetermined setting (i.e., a set breath type). In some embodiments, the set breath type is determined by the controller 110 and/or ventilator 100 based on ventilator and/or patient parameters. In other embodiments, the set breath type is a predetermined breath type that is automatically utilized by the ventilator 100 when a breath type is not input or selected the operator. In some embodiments, the set support setting is determined by the controller 110 and/or ventilator 100 automatically based on patient parameters, such as age, height, weight, ideal body weight, and etc., input or selected by the operator.

In some embodiments, the NPA module 118, TAPA module 119, effort module 115, trigger module 113, and/or the IM effort module 117 are part of the controller 110 as illustrated in FIG. 1. In other embodiments, the NPA module 118, TAPA module 119, effort module 115, trigger module 113, and/or the IM effort module 117 are part of the processor 116, pneumatic system 102, and/or a separate computing device in communication with the ventilator 100.

Initiation and execution of a NPA breath type, TANPA breath type, TAPA breath type or PA breath type has two operation prerequisites: (1) detection of an inspiratory trigger; and (2) determining and commanding target inspiration pressures to be delivered to the patient 150 during inspiration.

The effort module 115 estimates a patient effort. The effort module 115 estimates patient effort based at least on monitored flow from the last computational cycle (e.g., 5 milliseconds, 10 milliseconds, etc.) of ventilator. The computational cycle as used herein refers to a set time period for ventilator computation. For example, if the computational cycle is 5 milliseconds, after 20 milliseconds the ventilator will have performed desired computations 4 different times (every 5 milliseconds during the 20 millisecond time period). The effort module 115 continuously monitors the patient's instantaneous inspiratory flow and/or instantaneous net lung volume based on sensor output from the flow sensor and/or the pressure sensor in the last computational cycle. The instantaneous inspiratory flow and instantaneous net lung volume are indicators of the patient's inspiratory effort. These signals, together with ongoing estimates of the patient's lung compliance and lung/airway resistance and the Equation of Motion (Target Pressure(t)=Total Pressure(t)−Patient Muscle Pressure(t)), allow the ventilator to estimate/calculate a patient effort.

In some embodiments, the effort module 115 estimates patient effort by utilizing the following patient effort equations:

$$\begin{matrix} ( t ) = ( 1.0 - β ) [ \int {Q}_{p} d t + {Q}_{p} ] \\ = ( 1.0 - β ) [ {V}_{p} + {Q}_{p} ]\end{matrix}$$

[symbol]

is the estimated amount of inspiratory pressure provided by the patient's muscles. Total pressure delivered to the patient is [ [symbol] ∫Qₚdt+Qₚ [symbol] ], which is the sum of the pressure contributions by the patient ( [symbol] ) and the ventilator (Pᵥₑₙₜ or Target Pressure). t stands for time in the continuous domain. β is the support setting (i.e., percentage of total support to be contributed by the ventilator). [symbol] is estimated patient resistance. [symbol] is estimated patient elastance. Qₚ is the flow rate into the patient. V_{P} is the volume going into the patient and is also represented as ∫Qₚdt. In some embodiments, the ventilator 100, controller 110, and/or the effort module 115 determine a flow-assist setting (K_{f}) and/or a volume assist setting (K_{V}) based on the operator selected support setting (β). In other embodiments, the operator inputs the support setting (β) by inputting a flow-assist setting (K_{f}) and/or a volume assist setting (K_{V}). In some embodiments, K_{f}=K_{V}=β. The effort module 115 sends the estimated patient effort for each computational cycle to the TAPA module 119. In alternative embodiments, the effort module 115 utilizes any suitable known system or method for calculating patient effort, such as ieSync, a physical sensor, and/or a muscle activity monitor.

Based on the above patient effort equation, the transfer function from the estimated patient effort ( [symbol] ) to the target inspiration pressure (Pᵥₑₙₜ) is:

$$\frac{{P}_{vent} ( t )}{ ( t )} = \frac{β \cdot {G}_{vent} ( s ) \cdot \frac{ s +}{{R}_{P} s + {E}_{P}}}{1 - β \cdot {G}_{vent} ( s ) \cdot \frac{ s +}{{R}_{P} s + {E}_{P}}}$$

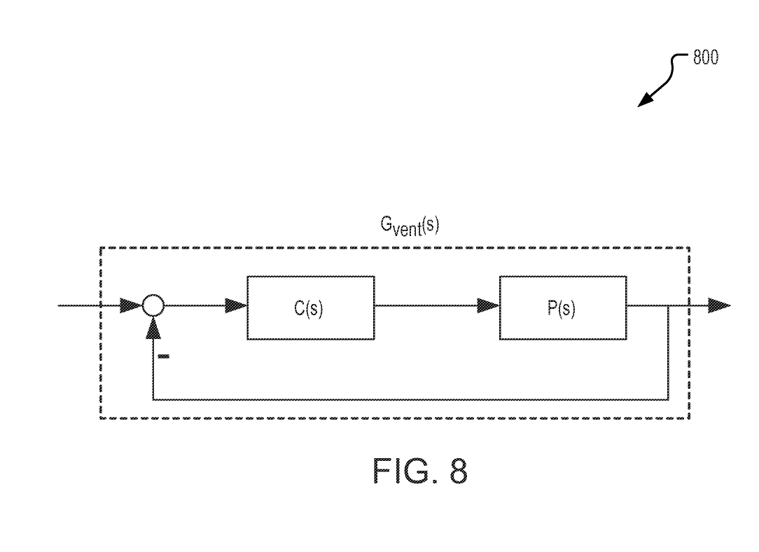

Gᵥₑₙₜ(s) represents pneumatic components of the ventilator with feedback controllers. s in Gᵥₑₙₜ(s) stands for the operator variable in the continuous-time domain. The transfer function Gᵥₑₙₜ(s) stands for the Laplace transform of a continuous-time function g(t). The transfer function shows that the closed-loop system in the conventional PA breath type and in TAPA breath type is a positive feedback system. R_{P} is patient resistance. E_{P} is patient elastance. s denotes a complex variable in an s-domain. FIG. 8 illustrates an embodiment of a ventilator control system, Gᵥₑₙₜ(s), scheme 800. A ventilator control system, Gᵥₑₙₜ(s), as illustrated in FIG. 8, can be written as:

$${G}_{vent} ( s ) = \frac{P ( s ) C ( s )}{1 + P ( s ) C ( s )}$$

C(s) represents a feedback controller, which can be a proportional integral derivative (PID) controller or lead-lag compensator. P(s) represents the pneumatic components to be controlled, e.g. flow valve, pressure valve, etc.

The trigger module 113 detects a patient initiated inspiratory trigger. The trigger module 113 continuously monitors flow and/or pressure based on sensor output from the flow sensor and/or the pressure sensor. In some embodiments, a patient trigger is determined by the trigger module 113 based on a measured or monitored patient inspiration flow and/or patient inspiration pressure. Any suitable type of triggering detection for determining a patient trigger may be utilized by the trigger module 113 of the ventilator 100, such as nasal detection, diaphragm detection, and/or brain signal detection. Further, the ventilator 100 and/or The trigger module 113 may detect patient triggering via a pressure-monitoring method, a flow-monitoring method, direct or indirect measurement of neuromuscular signals, or any other suitable method. Sensors 107 suitable for this detection may include any suitable sensing device as known by a person of skill in the art for a ventilator.

If the trigger module 113 detects a patient initiated trigger, the trigger module 113 sends instructions to the pressure generating system 102 to deliver the next breath. The pressure generating system 102 delivers a target inspiration pressure to the patient 150 during the next computational cycle based on instructions from the TAPA module 119. The next computational cycle is the computational cycle after the last computational cycle or after the most recent computational cycle. If the trigger module 113 does not detect a patient initiated trigger, the trigger module 113 continues to monitor for a patient initiated breath until a predetermined amount of time passes. If the trigger module 113 determines that the predetermined amount of time passes, the trigger module 113 sends instruction to the pressure generating system 102 to deliver the next breath.

The TAPA module 119 performs several functions. The TAPA module 119 receives a support setting. The support setting is received from user input or selection. For example, the user may input or select the support setting via a graphical user interface, wheel, mouse, or keyboard. If the support setting is not received from user input or selection, the TAPA module 119 receives a set support setting from the ventilator 100 and/or controller 110. In some embodiments, the set support setting is a predetermined support setting. In some embodiments, the set support setting is determined by the ventilator 100 and/or controller 110 based on patient parameters, such as height, weight, age, gender, and etc. As discussed above the support setting may be a percent or ratio of pressure support or may be a percent or ratio of volume support and flow support.

The TAPA module 119 receives the estimated patient effort for each computational cycle (e.g., 5 milliseconds, 10 milliseconds, etc.) from the effort module 115. The TAPA module 119 calculates a target pressure based at least on the received support setting, the estimated patient effort from the last computational cycle, and a time delay caused by a control system of the ventilator 100.

The time delay caused by the control system includes mechanical delay, electronic delay, software delay and/or pneumatic delay. The mechanical delay is a time lag caused by mechanical structures, such as the sensors measuring airway pressure and/or flow. Electronic delay is the time lag caused by electronic filters, such as the filter that reduce high-frequency noise in the measured signals. The software delay is the lag time caused by processors or microprocessors embedded in the ventilator. For example, the software delay may account for lag time cause by the processing of measured pressure and/or flow signals and/or processing an update to the target inspiration pressure for the next time point based on the control algorithm. Pneumatic delay is the lag time caused by pneumatic valves utilized to deliver a pressure and flow of breathing gas to a patient. Pneumatic valves generally need some amount of time to reach a desired pressure and/or flow, such as the target inspiration pressure. As a result, expiratory asynchrony due to control system time delay can result if the time delay caused by the control system is not accounted for in the calculation of the target pressure. Accordingly, the TAPA module 119 accounts for the time delay caused by the control system. For example, in some embodiments, the TAPA module 119 adjusts for the time delay caused by the control system by utilizing the following equation to calculate target inspiration pressure:

$${P}_{vent} ( t ) = {\overline{G}}_{vent} ( s ) \cdot β \cdot ( \frac{ s +}{{R}_{P} s + {E}_{P}} ) ( {P}_{vent} ( t - \hat{τ} ) + ( t - \hat{τ} ) )$$

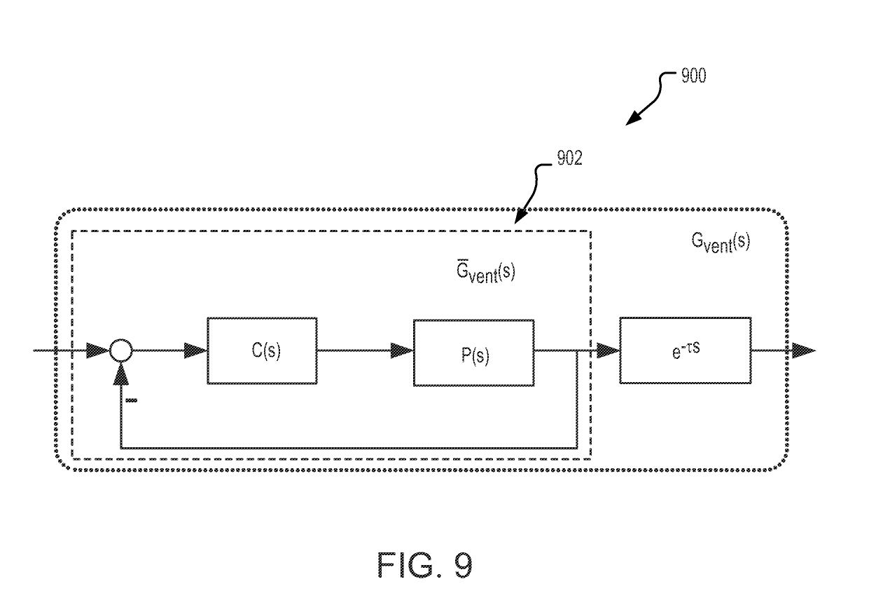

As discussed above, [symbol] is the estimated amount of inspiratory pressure provided by the patient's muscles. Pᵥₑₙₜ is the target inspiration pressure. t stands for time in the continuous domain. s denotes a complex variable in an s-domain. β is the support setting (i.e., percentage of total support to be contributed by the ventilator) input or selected by the operator. R_{P} is patient resistance. [symbol] is estimated patient resistance. E_{P} is patient elastance. [symbol] is estimated patient elastance. The support setting (β) is held constant over one breath. Every computational cycle (e.g., 5 milliseconds, 10 milliseconds, etc.), the ventilator calculates a target airway pressure, based on the received support setting, the time delay caused by the control system, and the patient effort received from the effort module 115. G̅ᵥₑₙₜ(s) is the transfer function representing dynamics of the control system with no delay. FIG. 9 illustrates an embodiment of a ventilator control system, Gᵥₑₙₜ(s), scheme 900. Scheme 900 includes the G̅ᵥₑₙₜ(s) 902. The estimated/measured time delay \hat{τ} and the estimated/measured lung flow Qₚ(t) and Qₚ(t−\hat{τ}) are used to calculate the dynamic pressure assist ratio

$$β \cdot \frac{{Q}_{p} ( t )}{{Q}_{p} ( t - \hat{τ} )}$$

to deal with the control system delay and improve the patient-ventilator interaction. \hat{τ} is an estimate of the control system time delay. \hat{τ}can be directly measured or indirectly estimated using a recursive algorithm. The example shown below is a direct measurement method.

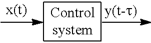

In a ventilator control system, the input x(t) is the calculated desired command; the output y(t−τ) (i.e., delivered pressure or flow) can be measured by a pressure and/or flow sensor. In software, with these two signals available, the time delay between the input x(t) and output y(t−τ) is calculated. The estimated time delay \hat{τ} is calculated as the timing difference between the two instants when x and y change in slope.

Once the TAPA module 119 determines the target inspiration pressure, the TAPA module 119 sends instructions to the pressure generating system 102 to deliver the calculated target inspiration pressure to the patient during the next computational cycle. As discussed above, The next computational cycle is the computational cycle after the last computational cycle or after the most recent computational cycle.

In some embodiments, the TAPA module 119 sends instructions to the pressure generating system 102 to deliver an initial inspiration pressure during a first computational cycle. The initial inspiration pressure is a predetermined pressure. In some embodiments, the initial inspiration pressure is a set pressure configured into the ventilator. In some embodiments, initial inspiration pressure varies based on patient parameters, such as age, height, weight, ideal body weight, and etc. In other embodiments, the initial inspiration pressure is set or selected by the operator. In some embodiments, the first computational cycle is the first computational cycle (e.g., the first 5 milliseconds, the first 10 milliseconds, etc.) of ventilating a patient 150 with a ventilator 100 after the ventilator is turned on. In some embodiments, the first computational cycle is the first computational cycle during a TAPA breath type delivered to a patient 150 by the ventilator. However, in alternative embodiments, the TAPA module 119 does not ever send instructions to deliver a predetermined initial inspiration pressure. In these embodiments, the TAPA module 119 only sends instructions to the pneumatic system to deliver the target inspiration pressure.

Positive feedback systems are not as stable as negative feedback systems. Accordingly, in some embodiments, the ventilator 100 includes an IM effort module 117 and a NPA module 118 that send instructions for delivering a NPA breath type or a TANPA breath type to a patient 150. The NPA and TANPA breath types are closed-loop systems of ventilation that are negative feedback systems.

The IM effort module 117 estimates a patient effort based at least on inspiratory flow monitored during the last computation cycle. The IM effort module 117 continuously monitors the patient's instantaneous inspiratory flow and/or instantaneous net lung volume based on sensor output from the flow sensor and/or the pressure sensor during the most recent computational cycle (i.e. last or most recent computational cycle). The instantaneous inspiratory flow and instantaneous net lung volume are indicators of the patient's inspiratory effort.

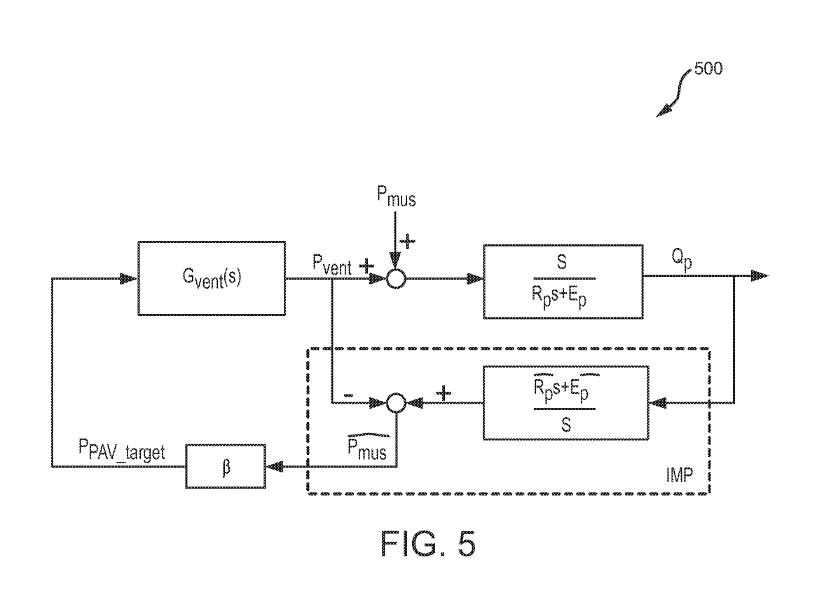

The IM effort module 117 estimates patient effort utilizing an inverse model principle (IMP). FIG. 5 illustrates the NPA breath type scheme 500 based on an injected inverse model principle (IMP). As shown in FIG. 5,

$$\frac{ s +}{s}$$

is the inverse model of the estimated respiratory system dynamics

$$\frac{s}{{R}_{P} s + {E}_{P}} .$$

As shown in FIG. 5, the input of the patient's respiratory system is disturbed by the patient's breathing effort (Pₘᵤₛ). In other words, Pₘᵤₛ is the input disturbance of the respiratory system. The inverse model principle states that disturbance Pₘᵤₛ can be estimated by utilizing feedback of the patient lung flow or the flow rate into the patient (Qₚ) and incorporating in the feedback path the inverse model of the estimated respiratory system dynamics. Based on FIG. 5 and the equation of motion, the flow rate into the patient (Qₚ) is shown in the flow equation below:

$${Q}_{P} = \frac{s}{{R}_{P} s + {E}_{P}} ( {P}_{mus} + {P}_{vent} )$$

By injecting Qₚ through the inverse model

$$( \frac{ s +}{s} )$$

and subtracting the target pressure (Pᵥₑₙₜ), the estimated muscle pressure (Pₘᵤₛ) is calculated based on the following effort equation:

$$( t ) = {Q}_{P} \frac{ s +}{s} - {P}_{vent}$$

Accordingly, the IM effort module 117 estimates patient effort by utilizing the above equation with the injected inverse model. As discussed above, [symbol] is the estimated amount of pressure provided by the patient's muscles or estimated patient effort, t is time in the continuous domain, Pᵥₑₙₜ is target inspiration pressure, [symbol] is estimated patient resistance, [symbol] is estimated patient elastance, and Qₚ is the flow rate into the patient. s denotes the complex variable in the s-domain.

The NPA module 118 performs several functions. The NPA module 118 receives a support setting. In some embodiments, the support setting is received from user input or selection. For example, the user may input or select the support setting via a graphical user interface, wheel, mouse, or keyboard. As discussed above the support setting may be a percent or ratio of pressure support or may be a percent or ratio of volume support and flow support. If a support setting is not received by the operator, the ventilator 100 may receive a predetermined support setting based on a set default setting and/or other patient parameters. In some embodiments, the ventilator 100, controller 110, and/or the NPA module 118 determine a flow-assist setting (K_{f}) and/or the volume assist setting (K_{V}) based on an operator selected support setting (β). In other embodiments, the operator inputs the support setting (β) by inputting a flow-assist setting (K_{f}) and/or a volume assist setting (K_{V}). In some embodiments, K_{f}=K_{V}=β.

The NPA module 118 receives the estimated patient effort for each computational cycle from the IM effort module 117. The NPA module 118 calculates a target pressure based at least on the received support setting and the estimated patient effort from the last computational cycle. In some embodiments, the NPA module 118 calculates a target pressure based on the received support setting and the estimated patient effort from the last computational cycle. Based on FIG. 5 and the equation of motion, in some embodiments, the NPA module 118 calculates a target inspiration pressure based on the following target inspiration pressure equation:

Pᵥₑₙₜ(t)=β· [symbol] (t)

Based on the above flow equation, effort equation, and target inspiration pressure equation for the NPA module 118, the transfer function from the estimated patient effort (Pₘᵤₛ) to the target inspiration pressure (Pᵥₑₙₜ) is:

$$\frac{{P}_{vent} ( t )}{ ( t )} = \frac{β \cdot {G}_{vent} ( s ) \frac{ s +}{{R}_{P} s + {E}_{P}}}{1 + β \cdot {G}_{vent} ( s ) \frac{ s +}{{R}_{P} s + {E}_{P}} [ \frac{{R}_{P} s + {E}_{P}}{ s +} - 1 ]} \qquad$$

The transfer function shows that the closed-loop system in the NPA breath type is a negative feedback system. The NPA module 118 transfer function is the closed-loop response of the NPA breath type scheme 500. Consequently, the steady-state value of

$$\frac{{P}_{vent} ( t )}{{P}_{mus} ( t )}$$

is obtained as shown in the steady state equations listed below:

$$\begin{matrix}{[ \frac{{P}_{vent} ( t )}{ ( t )} ]}_{t \to ∞} = {[ \frac{β \cdot {G}_{vent} ( s ) \frac{ s +}{{R}_{P} s + {E}_{P}}}{1 + β \cdot {G}_{vent} ( s ) \frac{ s +}{{R}_{P} s + {E}_{P}} [ \frac{{R}_{P} s + {E}_{P}}{ s +} - 1 ]} ]}_{s \to 0} \\ = \frac{β \cdot {G}_{vent} ( 0 ) \frac{{E}_{P}}{}}{1 + β \cdot {G}_{vent} ( 0 ) \frac{{E}_{P}}{} [ \frac{{E}_{P}}{} - 1 ]}\end{matrix}$$

The steady state equations shown above, imply that the identification of E_{P} is more critical in actual implementation, which is consistent with a traditional PA breath type. Assuming ideal conditions of Gᵥₑₙₜ(0)=1 and [symbol] =E_{P}, then the second steady state equation listed above becomes the following equation:

$${[ \frac{{P}_{vent} ( t )}{ ( t )} ]}_{t \to ∞} = β$$

The above equation shows that the objective of the NPA breath type scheme (linear amplification of the patient's effort) is obtained at a steady state unlike the conventional PA breath type. Accordingly, the closed-loop system of the NPA breath type delivered by the NPA module 118 is more stable than the closed-loop system of a conventional PA breath type. As shown by the NPA module 118 transfer function equation, the NPA breath type is a negative feedback system.

Negative feedback systems are more stable than positive feedback systems. Accordingly, the NPA breath type has a larger stability margin than the conventional PA breath type (see Example 1 below). Thus, the NPA breath type reduces and/or prevents “run-away” phenomenon when compared to the conventional PA breath type because the NPA breath type has a larger stability margin when compared to the conventional PA breath type. Additionally, the NPA breath type has better synchrony between the patient 150 and the ventilator 100 than the conventional PA breath type because the patient effort (Pₘᵤₛ) is estimated more directly and more accurately during the NPA breath type than in the conventional PA breath type. Accordingly, the ventilator support or the target inspiration pressure is more accurate in the NPA breath type than in the conventional PA breath type, which improves the synchrony between the ventilator 100 and the patient 150.

Identification of respiratory system resistance and elastance is significant during the NPA breath type. For example, both under and over estimates of resistance and elastance may significantly impair the synchrony between the patient and ventilator. Accordingly, in some embodiments, the IM effort module 117 and/or the NPA module 118 utilizes a recursive least square adaptive algorithm to estimate resistance and elastance. The recursive least square adaptive algorithm guarantees that estimated resistance and compliance asymptomatically converge to real values in the patient's respiratory system. Therefore, the IM effort module 117 and/or the NPA module 118 utilizing a recursive least square adaptive algorithm accurately estimates resistance and compliance improving synchrony between the ventilator and patient when compared to ventilators that do not utilize the recursive least square adaptive algorithm to estimate resistance and compliance. In some embodiments, the recursive least square adaptive algorithm is illustrated below:

$${\hat{θ}}^{T} ( k ) = {\hat{θ}}^{T} ( k - 1 ) + F ( k ) ϕ ( k ) e° ( k )$$

$$e° ( k ) = ( \frac{{P}_{vent} ( k )}{β} + {P}_{vent} ( k ) ) - {ϕ}^{T} ( k ) \hat{θ} ( k - 1 )$$

$$F ( k ) = F ( k - 1 ) - \frac{F ( k - 1 ) ϕ ( k ) {ϕ}^{T} ( k ) F ( k - 1 )}{1 + {ϕ}^{T} ( k ) F ( k - 1 ) ϕ ( k )}$$

where θ^{T}(k)=[R_{P}(k) E_{P}(k)] is the patient respiratory parameters to be estimated;

$$ϕ ( k ) = [ \begin{matrix}{Q}_{P} ( k ) \\ {V}_{P} ( k )\end{matrix} ]$$

is the regression parameter vector, which can be directly measured or indirectly calculated; \hat{θ}^{T}(k)=[ [symbol] (k) [symbol] (k)], which is the estimated patient respiratory parameter vector; and F(k)=F^{T}(k)>0 is the recursive least square gain at the computation cycle k.

Thus, an estimated resistance and elastance may be derived by the IM effort module 117 and/or the NPA module 118 using the recursive least square adaptive algorithm, as described above. Specifically, the parameter estimate vector update equation may solve for a recursive least squares gain value representing the resistance and elastance at a time instance based on a squared gain value for a previous time instance by subtracting a squared gain value for the previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the squared gain value for the previous time instance multiplied by the regression parameter vector at the time instance from a gain value for the previous time instance. The end result of the above calculation will provide an estimated resistance and elastance. In some embodiments, the recursive least square adaptive algorithm may be modified by introducing a forgetting factor 0<μ<1, such that the update equation becomes:

$$F ( k ) = \frac{1}{μ} [ F ( k - 1 ) - \frac{F ( k - 1 ) ϕ ( k ) {ϕ}^{T} ( k ) F ( k - 1 )}{μ + {ϕ}^{T} ( k ) F ( k - 1 ) ϕ ( k )} ]$$

In such instances, the closer μ is to 1, the less responsive the adaptive parameter estimation will be to parameter variations.

In some embodiments, the NPA module 118 during a TANPA breath type calculates a target pressure based on the time delay caused by the control system in addition to the estimated patient effort from the last computational cycle and the received support setting as discussed above. As discussed above, the time delay caused by the control system includes mechanical delay, electronic delay, software delay and/or pneumatic delay, each of which, are discussed in detail above. Accordingly, expiratory asynchrony due to control system time delay can result if the time delay caused by the control system is not accounted for in the calculation of the target pressure. Thus in some embodiments, the NPA module 118 accounts for the time delay caused by the control system. For example, in some embodiments, the NPA module 118 during a TANPA breath type adjusts for the time delay caused by the control system by utilizing the following equation to calculate the target inspiration pressure:

$${P}_{vent} ( t ) = {\overline{G}}_{vent} ( s ) \cdot {e}^{- \hat{τ} s} \cdot β \cdot \frac{ ( t )}{ ( t ) ( t - \hat{τ} )} ( t )$$

As discussed above, Pᵥₑₙₜ is a target inspiration pressure, [symbol] is estimated patient effort, t is time in the continuous domain, and β is a support setting Ĝᵥₑₙₜ(s) is the transfer function representing dynamics of the control system with no delay. The estimated or calculated time delay \hat{τ} and the estimated or measured lung flow Q_{P}(t) and Q_{pl (t-\hat{τ}) are used to calculate the dynamic pressure assist ratio}

$$β \cdot \frac{ ( t )}{ ( t ) ( t - \hat{τ} )}$$

to deal with the control system delay and improve the patient-ventilator interaction. e stands for the exponential function. \hat{τ} is an estimate of the control system time delay.

Once the NPA module 118 determines the target inspiration pressure, the NPA module 118 sends instructions to the pressure generating system 102 to deliver the calculated target inspiration pressure in the next computational cycle. As discussed above, the next computational cycle is the computational cycle after the last computational cycle or after the most recent computational cycle.

In some embodiments, the NPA module 118 sends instructions to the pressure generating system 102 to deliver a predetermined initial inspiration pressure during a first computational cycle. In some embodiments, the first computational cycle is the first computational cycle during ventilation of the patient 150 after the ventilator is turned on. In some embodiments, the first computational cycle is the first computational cycle of a NPA breath type delivered to a patient. However, in alternative embodiments, the NPA module 118 does not send instructions to deliver a predetermined initial inspiration pressure. In these embodiments, the NPA module 118 only sends instructions to the pneumatic system 102 to deliver the target inspiration pressure.

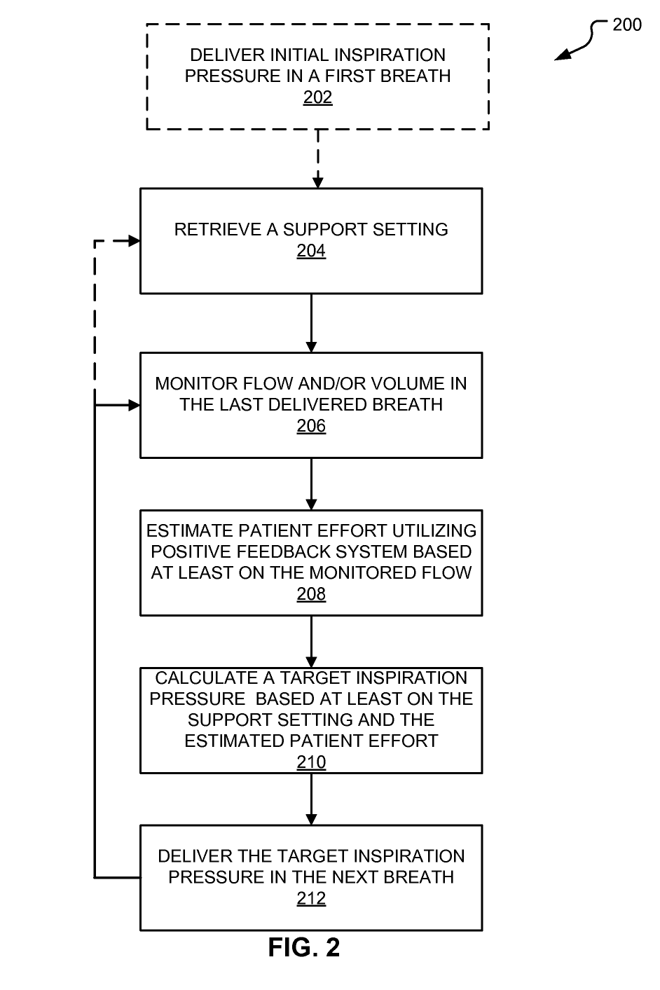

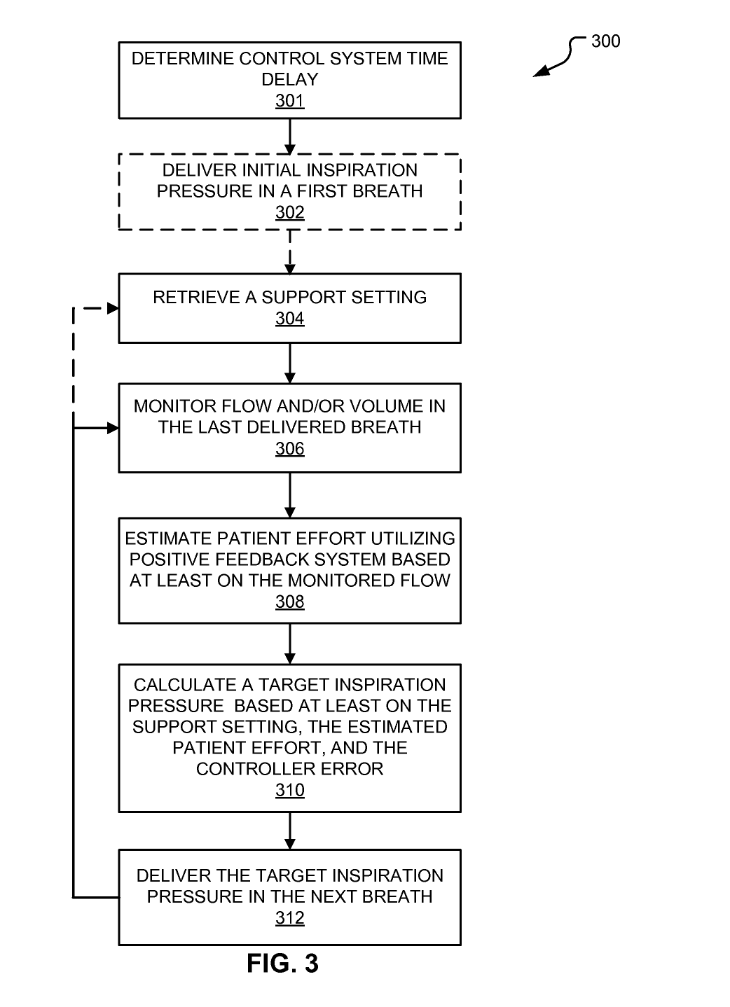

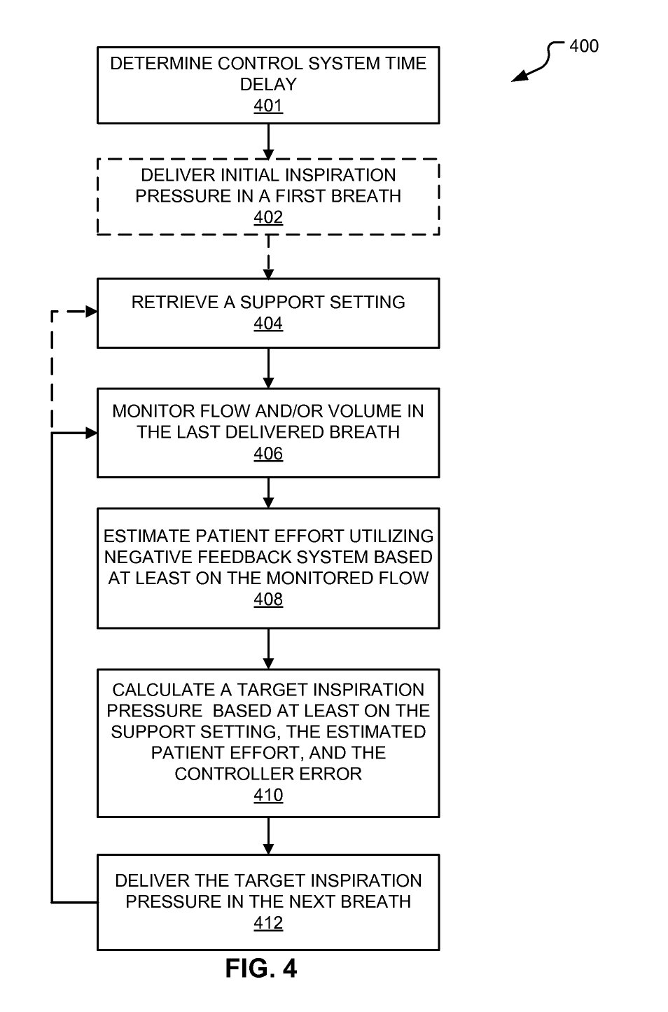

FIG. 2 illustrates an embodiment of a method 200 for ventilating a patient with ventilator utilizing a NPA breath type. FIG. 3 illustrates an embodiment of a method 300 for ventilating a patient with a ventilator utilizing a TANPA breath type. FIG. 4 illustrates an embodiment of a method 400 for ventilating a patient with a ventilator utilizing a TAPA breath type.

The PA, NPA, TANPA, and TAPA breath types each refer to a type of ventilation in which the ventilator or pressure generating system of the ventilator acts as an inspiratory amplifier that provides pressure support to the patient. The PA, NPA, TANPA, and TAPA breath types each deliver a target inspiration pressure calculated based on an estimated patient effort from the last computational cycle and a received support setting. However, the PA, NPA, TANPA, and TAPA breath types calculate the target inspiration pressure in different ways. The PA breath type determines a target pressure based on the following equation:

Target Airway Pressure(t)=β[ [symbol] ∫Qₚdt+Qₚ [symbol] ]

Target inspiration pressure (also referred to herein as “Pᵥₑₙₜ”) is the amount of pressure provided by the ventilator, total pressure delivered to the patient ([ [symbol] ∫Qₚdt+Qₚ [symbol] ]) or the sum of contributions by the patient and ventilator, and β is the support setting (i.e., percentage of total support to be contributed by the ventilator). In theory, with a PA breath type, the target pressure is proportional to the patient effort.

The TAPA breath type is similar to the PA breath type, but adjusts the above equation to for any time delay caused by a control system of the ventilator. As discussed above, the time delay caused by the control system includes mechanical delay, electronic delay, software delay and/or pneumatic delay each of which is discussed in detail above. In some embodiments, the TAPA breath type utilizes a dynamic assist (DAR) ratio to adjust for the time delay caused by the control system of the ventilator. Accordingly, in some embodiments, the TAPA breath type determines a target pressure based on the following equation:

$${P}_{vent} ( t ) = {\overline{G}}_{vent} ( s ) \cdot β \cdot ( \frac{ s +}{{R}_{P} s + {E}_{P}} ) ( {P}_{vent} ( t - \hat{τ} ) + ( t - \hat{τ} ) )$$

As discussed above, [symbol] is the estimated amount of inspiratory pressure provided by the patient's muscles. Pᵥₑₙₜ is the target inspiration pressure. t stands for time in the continuous domain. s denotes a complex variable in an s-domain. β is the support setting (i.e., percentage of total support to be contributed by the ventilator). R_{P} is patient resistance. [symbol] is estimated patient resistance. E_{P} is patient elastance. [symbol] is estimated patient elastance. The support setting (β) is held constant over one breath. \hat{τ} is an estimate of the control system time delay. G̅ᵥₑₙₜ(s) is the transfer function representing dynamics of the control system with no delay. The estimated/measured time delay \hat{τ} and the estimated/measured lung flow Qₚ(t) and Qₚ(t−\hat{τ}) are used to calculate the dynamic pressure assist ratio

$$β \cdot \frac{{Q}_{P} ( t )}{{Q}_{P} ( t - \hat{τ} )}$$

to deal with the control system delay and improve the patient-ventilator interaction.

The NPA breath type utilizes a negative feedback system based on an inverse model principle to estimate patient effort. The negative feedback system provides a more stable and more accurate estimate of patient effort and prevents or reduces the likelihood of a run-away when compared to the conventional PA breath type. This more stable estimated patient effort is then used to generate the target pressure of the ventilator. Because the estimate of patient effort is more accurate during the NPA breath type, so too, is the ventilator support, improving the synchrony between the ventilator and the patient during the NPA breath type when compared to the conventional PA breath type. The respiratory parameters (including resistance and elastance) are identified by using a recursive least square (RLS) based adaptive algorithm. The NPA breath type determines a target pressure based on the following equation:

Pᵥₑₙₜ(t)=β· [symbol] (t)

The TANPA breath type is similar to the NPA breath type and utilizes a negative feedback system based on an inverse model principle to estimate patient effort, but adjusts the above equation for any time delay caused by a control system of the ventilator. As discussed above, the time delay caused by the control system includes mechanical delay, electronic delay, software delay and/or pneumatic delay each of which is discussed in detail above. In some embodiments, the TANPA breath type utilizes a dynamic assist (DAR) ratio to adjust for the time delay caused by the control system of the ventilator. Accordingly, in some embodiments, the TANPA breath type determines a target pressure based on the following equation:

$${P}_{vent} ( t ) = {\overline{G}}_{vent} ( s ) \cdot {e}^{- τ s} \cdot β \cdot \frac{\hat{{P}_{mus}} ( t )}{\hat{{P}_{mus}} ( t ) ( t - \hat{τ} )} \hat{{P}_{mus}} ( t )$$

Additionally, the NPA and TANPA breath types estimate patient effort differently than the PA and TAPA breath types. The PA and TAPA breath types estimate patient effort utilizing the following equation:

[symbol]

(t)=(1.0−β)[Eₚ∫Qₚdt+QₚRₚ]

In contrast, the NPA and TANPA breath types estimate patient effort utilizing the inverse model principle (IMP). FIG. 5 illustrates the NPA breath type scheme 500 based on the injected inverse model principle (IMP). As shown in FIG. 5,

$$\frac{\hat{{R}_{P}} s + \hat{{E}_{P}}}{s}$$

is the inverse model of the estimated respiratory system dynamics

$$\frac{s}{{R}_{P} s + {E}_{P}} .$$

As shown in FIG. 5, the input of the patient's respiratory system is disturbed by the patient's breathing effort (Pₘᵤₛ). In other words, Pₘᵤₛ is the input disturbance of the respiratory system. The inverse model principle states that disturbance Pₘᵤₛ can be estimated by utilizing feedback of the patient lung flow or the flow rate into the patient (Qₚ) and incorporating in the feedback path the inverse model of the estimated respiratory system dynamics. Based on FIG. 5 and the equation of motion, the flow rate into the patient (Qₚ) is shown in the flow equation below:

$${Q}_{p} = \frac{s}{{R}_{P} s + {E}_{P}} ( \hat{{P}_{mus}} + {P}_{vent} )$$

By injecting Q through the inverse model

$$( \frac{\hat{{R}_{P}} s + \hat{{E}_{P}}}{s} )$$

and subtracting the target pressure (Pᵥₑₙₜ), the estimated muscle pressure ( [symbol] ) is calculated based on the following effort equation:

$$\hat{{P}_{mus}} ( t ) = {Q}_{p} \frac{\hat{{R}_{P}} s + \hat{{E}_{P}}}{s} - {P}_{vent}$$

Accordingly, the NPA and TANPA breath types estimate patient effort by utilizing the above equation with the injected inverse model. As discussed above, [symbol] is the estimated amount of pressure provided by the patient's muscles, Pᵥₑₙₜ is target inspiration pressure, t is time in the continuous domain, [symbol] is estimated patient resistance, [symbol] is estimated patient elastance, and Q_{P} is the flow rate into the patient. s denotes the complex variable in the s-domain.

FIG. 2 illustrates an embodiment of a method 200 for ventilating a patient with a ventilator utilizing a NPA breath type. As illustrated, method 200 includes a retrieving operation 204. The ventilator during a retrieving operation 204, retrieves a support setting. The support setting is the percentage or ratio of total support to be contributed by the ventilator. In some embodiments, the support setting is divided into a flow-assist setting (K_{f}) and a volume-assist setting (K_{V}).

In some embodiments, the ventilator during a retrieving operation 204 retrieves the support setting from operator input or selection. In some embodiments, the ventilator during a retrieving operation 204 retrieves the support setting from a determination made automatically by the controller and/or ventilator based on ventilator and/or patient parameters. In further embodiments, the ventilator during a retrieving operation 204 retrieves the support setting from a predetermined setting that is automatically utilized by the ventilator when a support setting is not input or selected by the operator. In some embodiments, the ventilator during a retrieving operation 204 determines a flow-assist setting and a volume assist setting based on an operator selected support setting. In other embodiments, the ventilator during a retrieving operation 204 retrieves the support setting from an operator selected or input flow-assist setting and volume assist setting.

Method 200 also includes a monitoring operation 206. The ventilator during the monitoring operation 206 monitors at least inspiration flow during a computational cycle. The ventilator during the monitoring operation 206 may also monitor the net lung volume during the computational cycle based at least on the monitored inspiration flow. The ventilator during the monitoring operation 206 monitors the inspiration flow during a computational cycle utilizing a sensor, such as a flow sensor and/or pressure sensor. The inspiratory flow and net lung volume are indicators of the patient's inspiratory effort.

Further, method 200 includes an estimating operation 208. The ventilator, controller, and/or IM effort module during the estimating operation 208 estimates a patient effort for the last computational cycle. The ventilator, controller, and/or IM effort module during the estimating operation 208 estimates a patient effort utilizing an inverse model based at least on the monitored inspiration flow from the last computational cycle. As discussed above, method 200 delivers ventilation according to the NPA breath type, which is discussed in detail above. Accordingly, in some embodiments, the ventilator, controller, and/or IM effort module, during the estimating operation 208, estimate muscle pressure (Pₘᵤₛ) or patient effort utilizing the following effort equation:

$$\hat{{P}_{mus}} ( t ) = {Q}_{p} \frac{\hat{{R}_{P}} s + \hat{{E}_{P}}}{s} - {P}_{vent}$$

Identification of respiratory system resistance and elastance is significant during the NPA breath type. For example, both under an over estimates of resistance and elastance may significantly impair the synchrony between the patient and ventilator. Accordingly, in some embodiments, the ventilator and/or the IM effort module during the estimating operation 208 utilizes a recursive least square adaptive algorithm to estimate resistance and elastance. The recursive least square adaptive algorithm guarantees that estimated resistance and compliance asymptomatically converge to real values in the patient's respiratory system. Therefore, the ventilator and/or the IM effort module utilizing a recursive least square adaptive algorithm during the estimating operation 208 accurately estimate resistance and compliance improving synchrony between the ventilator and patient when compared to ventilators that do not utilize the recursive least square adaptive algorithm to estimate resistance and compliance. In some embodiments, the recursive least square adaptive algorithm is illustrated below:

$${\hat{θ}}^{T} ( k ) = {\hat{θ}}^{T} ( k - 1 ) + F ( k ) ϕ ( k ) e ° ( k )$$

$$e ° ( k ) = ( \frac{{P}_{vent} ( k )}{β} + {P}_{vent} ( k ) ) - {ϕ}^{T} ( k ) \hat{θ} ( k - 1 )$$

$$F ( k ) = F ( k - 1 ) - \frac{F ( k - 1 ) ϕ ( k ) {ϕ}^{T} ( k ) F ( k - 1 )}{1 + {ϕ}^{T} ( k ) F ( k - 1 ) ϕ ( k )}$$

where θ^{T}(k)=[R_{P}(k) E_{P}(k)] is the patient respiratory parameters to be estimated;

$$ϕ ( k ) = [ \begin{matrix}{Q}_{p} ( k ) \\ {V}_{p} ( k )\end{matrix} ]$$

is the regression parameter vector, which can be directly measured or indirectly calculated; θ^{T}(k)=[ [symbol] (k) [symbol] (k)], which is the estimated patient respiratory parameter vector; and F(k)=F^{T}(k)>0 is the recursive least square gain at the computation cycle k.

Thus, an estimated resistance and elastance may be derived by the ventilator and/or the IM effort module during the estimating operation 208 using the recursive least square adaptive algorithm, as described above. Specifically, the parameter estimate vector update equation may solve for a recursive least squares gain value representing the resistance and elastance at a time instance based on a squared gain value for a previous time instance by subtracting a squared gain value for the previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the squared gain value for the previous time instance multiplied by the regression parameter vector at the time instance from a gain value for the previous time instance. The end result of the above calculation will provide an estimated resistance and elastance. In some embodiments, the recursive least square adaptive algorithm may be modified by introducing a forgetting factor 0<μ<1, such that the update equation becomes:

$$F ( k ) = \frac{1}{μ} [ F ( k - 1 ) - \frac{F ( k - 1 ) ϕ ( k ) {ϕ}^{T} ( k ) F ( k - 1 )}{μ + {ϕ}^{T} ( k ) F ( k - 1 ) ϕ ( k )} ]$$

In such instances, the closer μ is to 1, the less responsive the adaptive parameter estimation will be to parameter variations.

As illustrated, method 200 includes a calculating operation 210. During calculating operation 210, the ventilator, controller, and/or NPA module, calculates a target inspiration pressure. During calculating operation 210, the ventilator, controller, and/or NPA module, calculates a target inspiration pressure based at least on the estimated patient effort from the last computational cycle and the received support setting. As discussed above, method 200 delivers ventilation according to the NPA breath type, which is discussed in detail above. Accordingly, in some embodiments, the ventilator, controller, and/or NPA module, during the calculating operation 210, calculate a target inspiration pressure (Pᵥₑₙₜ) utilizing the following effort equation:

Pᵥₑₙₜ(t)=β· [symbol] (t)

Next, method 200 includes a delivering operation 212. During delivering operation 212, the ventilator and/or the pressure generating system deliver the target inspiration pressure to the patient in the next computational cycle. The ventilator and/or the pressure generating system may deliver the target inspiration pressure by adjusting the flow and/or pressure of the delivered gas to the patient. In some embodiments, the ventilator and/or the pressure generating system adjusts the pressure and/or flow of the delivered gas by adjusting one or more valves, such as a solenoid valve, between the compressor or another source of pressurized gases and the patient.

As the ventilator performs the delivering operation 212, the ventilator performs the monitoring operation 206 again as described above. Method 200 performs the monitoring operation 206, estimating operation 208, calculating operation 210, and delivering operation 212 repeatedly creating a closed-loop system of ventilation. In some embodiments, the ventilator during method 200 also performs the retrieving operation 204 repeatedly with operations (206, 208, 210, and 212) listed above. In embodiments where the support setting is input or selected by the operator, the retrieving operation 204 will retrieve the same support setting until an operator inputs or selects a new support setting. In other embodiments where the support setting is determined by the ventilator based on patient parameters, the retrieving operation 204 will retrieve the same support setting until an operator inputs or selects new patient parameters. Further, the ventilator, IM effort module, and/or controller during the estimating operation 208 estimates a new patient effort or updates the estimated patient effort after each computational cycle, such as the first, second, third, and etc. computational cycles during the NPA breath type. The new or updated patient effort may be the same or different from the previous estimated patient efforts. Similarly, the ventilator, NPA module, and/or controller during the calculating operation 210 calculates a new target inspiration pressure or updates the target inspiration pressure after each computational cycle, such as the first, second, third, and etc. computational cycles during the NPA breath type. The new or updated target inspiration pressure may be the same or different from the previously calculated target inspiration pressures.

This closed-loop system of ventilation is a negative feedback system. Based on the above flow equation, effort equation, and target inspiration pressure equation for the NPA breath type, the transfer function from the patient effort (Pₘᵤₛ) to the target inspiration pressure (Pᵥₑₙₜ) is:

$$\frac{{P}_{vent} ( t )}{\hat{{P}_{mus}} ( t )} = \frac{β \cdot {G}_{vent} ( s ) \frac{\hat{{R}_{P}} s + \hat{{E}_{P}}}{{R}_{P} s + {E}_{P}}}{1 + β \cdot {G}_{vent} ( s ) \frac{\hat{{R}_{P}} s + \hat{{E}_{P}}}{{R}_{P} s + {E}_{P}} [ \frac{{R}_{P} s + {E}_{P}}{\hat{{R}_{P}} s + \hat{{E}_{P}}} - 1 ]}$$

The transfer function shows that the closed-loop system in the NPA breath type is a negative feedback system. The transfer function is the closed-loop response of the NPA breath type scheme 500 as illustrated in FIG. 5. Consequently, the steady-state value of

$$\frac{{P}_{vent} ( t )}{{P}_{mus} ( t )}$$

is obtained as shown in the steady state equations listed below:

$$\begin{matrix}{[ \frac{{P}_{vent} ( t )}{\hat{{P}_{mus}} ( t )} ]}_{t -> ∞} = {[ \frac{β \cdot {G}_{vent} ( s ) \frac{\hat{{R}_{P}} s + \hat{{E}_{P}}}{{R}_{P} s + {E}_{P}}}{1 + β \cdot {G}_{vent} ( s ) \frac{\hat{{R}_{P}} s + \hat{{E}_{P}}}{{R}_{P} s + {E}_{P}} [ \frac{{R}_{P} s + {E}_{P}}{\hat{{R}_{P}} s + \hat{{E}_{P}}} - 1 ]} ]}_{s -> 0} \\ = \frac{β \cdot {G}_{vent} ( 0 ) \frac{\hat{{E}_{P}}}{{E}_{P}}}{1 + β \cdot {G}_{vent} ( 0 ) \frac{\hat{{E}_{P}}}{{E}_{P}} [ \frac{{E}_{P}}{\hat{{E}_{P}}} - 1 ]}\end{matrix}$$

The steady state equations shown above, imply that the identification of E_{P} is more critical in actual implementation, which is consistent with a traditional PA breath type. Assuming ideal conditions of Gᵥₑₙₜ(0)=1 and [symbol] =E_{P}, then the second steady state equation listed above becomes the following equation:

$${[ \frac{{P}_{vent} ( t )}{\hat{{P}_{mus}} ( t )} ]}_{t -> ∞} = β$$

The above equation shows that the objective of the NPA breath type scheme (linear amplification of the patient's effort) is obtained at a steady state unlike the conventional PA breath type. Accordingly, the closed-loop system of the NPA breath type is more stable than the closed-loop system in a conventional PA breath type. As shown by the above transfer function equation, the NPA breath type is a negative feedback system when E_{P} and R_{P} are accurately identified. Negative feedback systems are more stable than positive feedback systems. Accordingly, the NPA breath type has a larger stability margin when compared to the conventional PA breath type (see Example 1 below). Thus, the NPA breath type reduces and/or prevents “run-away” phenomenon when compared to the conventional PA breath type because the NPA breath type has a larger stability margin than the conventional PA breath type. Additionally, the NPA breath type has better synchrony between the patient and the ventilator than the conventional PA breath type because patient effort (Pₘᵤₛ) is estimated more directly and more accurately in the NPA breath type than in the conventional PA breath type. Accordingly, the ventilator support or target inspiration pressure is more accurate in the NPA breath type than in the conventional PA breath type, which improves the synchrony between the ventilator and the patient.

In some embodiments, method 200 includes an initial delivering operation 202. The ventilator and/or pressure generating system during the initial delivering operation 202 delivers an initial inspiration pressure to the patient during a first computational cycle. In this embodiment, the first computational cycle is the first computational cycle during ventilation of a patient after the ventilator is switched on and/or is the first computational cycle during a NPA breath type. The initial inspiration pressure is a predetermined pressure. In some embodiments, the initial inspiration pressure is a set pressure configured into the ventilator. In some embodiments, the initial inspiration pressure varies based on patient parameters, such as age, height, weight, ideal body weight, and etc. In other embodiments, the initial inspiration pressure is set or selected by the operator. However, in embodiments where method 200 does not include an initial delivering operation 202, the ventilator and/or pressure generating system only deliver the target inspiration pressure to the patient during method 200.

In some embodiments, method 200 includes a displaying operation. The ventilator during the displaying operation displays any suitable information for display on a ventilator. In one embodiment, the displaying operation displays one or more of the breath type, the estimated patient effort, the calculated target pressure, the total pressure delivered, the monitored inspiration pressure, the monitored net lung volume, an initial inspiratory pressure, a list of delivered target inspiration pressures for a predetermined number of computational cycles, a list of estimated patient efforts from a predetermined number of computational cycles, a graph of the list of the delivered target inspiration pressure and/or the estimated patient efforts for a predetermined number of computational cycles, the support setting, a volume-assist setting, and/or a flow-assist setting.

FIG. 3 illustrates an embodiment of a method 300 for ventilating a patient with a ventilator utilizing a TANPA breath type. As illustrated, method 300 includes a retrieving operation 304. The retrieving operation 304 is similar to retrieving operation 204 described above. The ventilator during a retrieving operation 304, retrieves a support setting. The support setting is the percentage or ratio of total support to be contributed by the ventilator. In some embodiments, the support setting is a flow-assist setting (K_{f}) and/or a volume-assist setting (K_{V}).

In some embodiments, the ventilator during a retrieving operation 304 retrieves the support setting from operator input or selection. In some embodiments, the ventilator during a retrieving operation 304 retrieves the support setting from a determination made automatically by the controller and/or ventilator based on patient parameters, such as age, height, weight, gender, ideal body weight, and etc. In further embodiments, the ventilator during a retrieving operation 304 retrieves the support setting from a predetermined setting that is automatically utilized by the ventilator when a support setting is not input or selected by the operator. In some embodiments, the ventilator during a retrieving operation 304 determines a flow-assist setting and/or a volume assist setting based on an operator selected support setting. In other embodiments, the ventilator during a retrieving operation 304 retrieves the support setting from an operator selected or input flow-assist setting and/or volume assist setting.

Additionally, method 300 includes a determining operation 301. The ventilator and/or controller during the determining operation 301 determine a time delay caused by a control system. The control system as used herein refers to any portions of the ventilator that are utilized to control the gas delivery of the ventilator, such as a controller, valve, inspiratory module, expiratory module, flow sensor, pressure sensor, and/or software. The time delay caused by the control system includes mechanical delay, electronic delay, software delay and/or pneumatic delay, which is discussed above in further detail. As a result, expiratory asynchrony due to control system time delay can result if the time delay caused by the control system is not accounted for in the calculation of the target pressure. Accordingly, the ventilator and/or controller during determining operation 301 determine the time delay caused by the control system. In some embodiments, a test breath is ran on a ventilator utilizing a fake lung or patient in which actual response times for mechanical delay, electronic delay, software delay and/or pneumatic delay are calculated.

Method 300 also includes a monitoring operation 306, which is similar to the monitoring operation 206 described above. The ventilator during the monitoring operation 306 monitors at least inspiration flow during a computational cycle. The ventilator during the monitoring operation 306 may also monitor the net lung volume during a computational cycle based at least on the monitored inspiration flow. The ventilator during the monitoring operation 306 monitors the inspiration flow during a computational cycle utilizing a sensor, such as a flow sensor and/or pressure sensor. The inspiratory flow and net lung volume are indicators of the patient's inspiratory effort.

Further, method 300 includes an estimating operation 308, which is similar to the estimating operation 208 described above. The ventilator, controller, and/or IM effort module during the estimating operation 308 estimates a patient effort for the last computational cycle. The ventilator, controller, and/or IM effort module during the estimating operation 308 estimates a patient effort utilizing an inverse model based at least on the monitored inspiration flow from the last computational cycle. As discussed above, method 300 delivers ventilation according to the TANPA breath type, which is discussed in detail above. Accordingly, in some embodiments, the ventilator, controller, and/or IM effort module, during the estimating operation 308, estimate muscle pressure (Pₘᵤₛ) or patient effort utilizing the following effort equation:

$$\hat{{P}_{mus}} ( t ) = {Q}_{p} \frac{\hat{{R}_{P}} s + \hat{{E}_{P}}}{s} - {P}_{vent}$$

Identification of respiratory system resistance and elastance is significant during the TANPA breath type. For example, both under an over estimates of resistance and elastance may significantly impair the synchrony between the patient and ventilator. Accordingly, in some embodiments, the ventilator and/or the IM effort module during the estimating operation 308 utilize a recursive least square adaptive algorithm to estimate resistance and elastance. The recursive least square adaptive algorithm guarantees that estimated resistance and compliance asymptomatically converge to real values in the patient's respiratory system. Therefore, the ventilator and/or the IM effort module utilizing a recursive least square adaptive algorithm during the estimating operation 308 accurately estimate resistance and compliance improving synchrony between the ventilator and patient when compared to ventilators that do not utilize the recursive least square adaptive algorithm to estimate resistance and compliance. In some embodiments, the recursive least square adaptive algorithm is illustrated below:

$${\hat{θ}}^{T} ( k ) = {\hat{θ}}^{T} ( k - 1 ) + F ( k ) ϕ ( k ) e ° ( k )$$

$$e ° ( k ) = ( \frac{{P}_{vent} ( k )}{β} + {P}_{vent} ( k ) ) - {ϕ}^{T} ( k ) \hat{θ} ( k - 1 )$$

$$F ( k ) = F ( k - 1 ) - \frac{F ( k - 1 ) ϕ ( k ) {ϕ}^{T} ( k ) F ( k - 1 )}{1 + {ϕ}^{T} ( k ) F ( k - 1 ) ϕ ( k )}$$

where θ^{T}(k)=[R_{P}(k) E_{P}(k)] is the patient respiratory parameters to be estimated;

$$ϕ ( k ) = [ \begin{matrix}{Q}_{p} ( k ) \\ {V}_{p} ( k )\end{matrix} ]$$

is the regression parameter vector, which can be directly measured or indirectly calculated; θ^{T}(k)=[ [symbol] (k) [symbol] (k)], which is the estimated patient respiratory parameter vector; and F(k)=F^{T}(k)>0 is the recursive least square gain at the computation cycle k.

Thus, an estimated resistance and elastance may be derived by the ventilator and/or the IM effort module during the estimating operation 308 using the recursive least square adaptive algorithm, as described above. Specifically, the parameter estimate vector update equation may solve for a recursive least squares gain value representing the resistance and elastance at a time instance based on a squared gain value for a previous time instance by subtracting a squared gain value for the previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the squared gain value for the previous time instance multiplied by the regression parameter vector at the time instance from a gain value for the previous time instance. The end result of the above calculation will provide an estimated resistance and elastance. In some embodiments, the recursive least square adaptive algorithm may be modified by introducing a forgetting factor 0<μ<1, such that the update equation becomes:

$$F ( k ) = \frac{1}{μ} [ F ( k - 1 ) - \frac{F ( k - 1 ) ϕ ( k ) {ϕ}^{T} ( k ) F ( k - 1 )}{μ + {ϕ}^{T} ( k ) F ( k - 1 ) ϕ ( k )} ]$$

In such instances, the closer μ is to 1, the less responsive the adaptive parameter estimation will be to parameter variations.

As illustrated, method 300 includes a calculating operation 310. During calculating operation 310, the ventilator, controller, and/or NPA module, calculates a target inspiration pressure. During calculating operation 310, the ventilator, controller, and/or NPA module, calculates a target inspiration pressure based at least on the time delay caused the control system, the estimated patient effort from the last computational cycle and the received support setting. As discussed above, method 300 delivers ventilation according to the TANPA breath type, which is discussed in detail above. Accordingly, the ventilator, controller, and/or TANPA module, during the calculating operation 310, calculate a target inspiration pressure (Pᵥₑₙₜ) utilizing a target pressure equation that has been adjusted with a dynamic assist ratio. In some embodiments, the ventilator, controller, and/or NPA module, during the calculating operation 310, calculate a target inspiration pressure (Pᵥₑₙₜ) utilizing the following effort equation:

$${P}_{vent} ( t ) = {\overline{G}}_{vent} ( s ) \cdot {e}^{- τ s} \cdot β \cdot \frac{ ( t )}{ ( t ) ( t - \hat{τ} )} ( t )$$

wherein Ĝᵥₑₙₜ(s) is the transfer function representing dynamics of the control system with no delay. The estimated/measured time delay \hat{τ} and the estimated/measured lung flow Q_{P}(t) and Q_{L}(t−\hat{τ}) are used to calculate the dynamic pressure assist ratio

$$β \cdot \frac{ ( t )}{ ( t ) ( t - \hat{τ} )}$$

to deal with the control system delay and improve the patient-ventilator interaction. e stands for the exponential function and \hat{τ} is an estimate of the control system delay τ.

Next, method 300 includes a delivering operation 312. During delivering operation 312, the ventilator and/or the pressure generating system deliver the target inspiration pressure to the patient in the next computational cycle. The ventilator and/or the pressure generating system may deliver the target inspiration pressure by adjusting the flow and/or pressure of the delivered gas to the patient. In some embodiments, the ventilator and/or the pressure generating system adjusts the pressure and/or flow of the delivered gas by adjusting one or more valves, such as a solenoid valve, between the compressor or another source of pressurized gases and the patient.

As the ventilator performs the delivering operation 312, the ventilator performs the monitoring operation 306 again as described above. Method 300 performs the monitoring operation 306, estimating operation 308, calculating operation 310, and delivering operation 312 repeatedly creating a closed-loop system of ventilation. In some embodiments, the ventilator during method 300 also performs the retrieving operation 304 repeatedly with the operations (306, 308, 310, and 312) listed above. In embodiments where the support setting is input or selected by the operator, the retrieving operation 304 will retrieve the same support setting until an operator inputs or selects a new support setting. In embodiments where the support setting is determined by the ventilator based on input or selected patient parameters, the retrieving operation 304 will retrieve the same support setting until an operator inputs or selects new patient parameters. Further, the ventilator, IM effort module, and/or controller during the estimating operation 308 estimates a new patient effort or updates the estimated patient effort after each computational cycle, such the first, second, third, and etc. computational cycles during the TANPA breath type. The new or updated patient effort may be the same or different from the previous estimated patient efforts. Similarly, the ventilator, TANPA module, and/or controller during the calculating operation 310 calculates a new target inspiration pressure or updates the target inspiration pressure after each computational cycle, such the first, second, third, and etc. computational cycles during the TANPA breath type. The new or updated target inspiration pressure may be the same or different from the previously calculated target inspiration pressures.

This closed-loop system of ventilation is a negative feedback system. Based on the above flow equation, effort equation, and target inspiration pressure equation for the TANPA breath type, the transfer function from the patient effort (Pₘᵤₛ) to the target inspiration pressure (Pᵥₑₙₜ) is:

$$\frac{ ( t )}{{P}_{mus} ( t )} = \frac{β \cdot {G}_{vent} ( s ) \frac{ s +}{{R}_{P} s + {E}_{P}}}{1 + β \cdot {G}_{vent} ( s ) \frac{ s +}{{R}_{P} s + {E}_{P}} [ \frac{{R}_{P} s + {E}_{P}}{ s +} - 1 ]}$$

The transfer function shows that the closed-loop system in the TANPA breath type is a negative feedback system. The transfer function is the closed-loop response of the TANPA breath type scheme 500 as illustrated in FIG. 5. Consequently, the steady-state value of

$$\frac{{P}_{vent} ( t )}{{P}_{mus ( t )}}$$

is obtained as shown in the steady state equations listed below:

$$\begin{matrix}{[ \frac{{P}_{vent} ( t )}{ ( t )} ]}_{t -> ∞} = {[ \frac{β \cdot {G}_{vent} ( s ) \frac{ s +}{{R}_{P} s + {E}_{P}}}{1 + β \cdot {G}_{vent} ( s ) \frac{ s +}{{R}_{P} s + {E}_{P}} [ \frac{{R}_{P} s + {E}_{P}}{ s +} - 1 ]} ]}_{s -> 0} \\ = \frac{β \cdot {G}_{vent} ( 0 ) \frac{{E}_{P}}{}}{1 + β \cdot {G}_{vent} ( 0 ) \frac{{E}_{P}}{} [ \frac{{E}_{P}}{} - 1 ]}\end{matrix}$$

The steady state equation shown above, implies that the identification of E_{P} is more critical in actual implementation, which is consistent with a traditional PA breath type. Assuming ideal conditions of Gᵥₑₙₜ(0)=1 and [symbol] =E_{P}, then the second steady state equation listed above becomes the following equation:

$${p [ \frac{{P}_{vent} ( t )}{ ( t )} ]}_{t -> ∞} = β$$

The above equation shows that the objective of the TANPA breath type scheme (linear amplification of the patient's effort) is obtained at a steady state unlike the conventional PA breath type. Accordingly, the closed-loop system of the TANPA breath type is more stable than the closed-loop system in a conventional PA breath type. As shown by the above transfer function equation, the TANPA breath type is a negative feedback system when E_{P} and R_{P} are accurately identified. Negative feedback systems are more stable than positive feedback systems. Accordingly, the TANPA breath type has a larger stability margin when compared to the conventional PA breath type. Thus, the TANPA breath type reduces and/or prevents “run-away” phenomenon when compared to the conventional PA breath type because the TANPA breath type has a larger stability margin than the conventional PA breath type. Additionally, the TANPA breath type improves synchrony between the patient and the ventilator when compared to the conventional PA breath type because patient effort (Pₘᵤₛ) is estimated more directly and more accurately than in the conventional PA breath type. Accordingly, the ventilator support or target pressure is more accurate in the TANPA breath type when compared to the conventional PA breath type, which improves the synchrony between the ventilator and the patient.

In some embodiments, method 300 includes an initial delivering operation 302, which is similar to the initial delivering operation 202 described above. The ventilator and/or pressure generating system during the initial delivering operation 302 delivers an initial inspiration pressure to the patient during a first computational cycle. In this embodiment, the first computational cycles is the first computational cycle during ventilation of a patient after the ventilator is switched on and/or is the first computational cycle during the TANPA breath type. The initial inspiration pressure is a predetermined pressure. In some embodiments, the initial inspiration pressure is a set pressure configured into the ventilator. In some embodiments, the initial inspiration pressure varies based on patient parameters, such as age, height, weight, ideal body weight, and etc. In other embodiments, the initial inspiration pressure is set or selected by the operator. However, in embodiments where method 300 does not include an initial delivering operation 302, the ventilator and/or pressure generating system only deliver the target inspiration pressure to the patient during method 300.

In some embodiments, method 300 includes a displaying operation. The ventilator during the displaying operation displays any suitable information for display on a ventilator. In one embodiment, the displaying operation displays one or more of the breath type, the estimated patient effort, the calculated target pressure, the total pressure delivered, the monitored inspiration pressure, the monitored net lung volume, an initial inspiratory pressure, a list of delivered target inspiration pressures for a predetermined number of computational cycles, a list of estimated patient efforts from a predetermined number of computational cycles, a graph of the list of the delivered target inspiration pressure and/or the estimated patient efforts for a predetermined number of computational cycles or an average or other function thereof, the support setting, a volume-assist setting, a flow-assist setting, and/or a time delay caused by the control system.

FIG. 4 illustrates an embodiment of a method 400 for ventilating a patient with a ventilator utilizing a TAPA breath type. As illustrated, method 400 includes a retrieving operation 404. The ventilator and/or controller during the retrieving operation 404 retrieve a support setting. The retrieving operation 404 is similar to retrieving operation 304 described above.

Additionally, method 400 includes a determining operation 401. The ventilator and/or controller during the determining operation 401 determine a time delay caused by a control system. The determining operation 401 is similar to the determining operation 301 of method 300 described above.

Method 400 also includes a monitoring operation 406, which is similar to the monitoring operation 306 described above. The ventilator during the monitoring operation 406 monitors at least inspiration flow during a computational cycle.

Further, method 400 includes an estimating operation 408. The ventilator, controller, and/or effort module during the estimating operation 408 estimates a patient effort for the last computational cycle. As discussed above, method 400 delivers ventilation according to the TAPA breath type, which is discussed in detail above. Accordingly, in some embodiments, the ventilator, controller, and/or effort module, during the estimating operation 408, estimate muscle pressure ( [symbol] ) or patient effort utilizing the following effort equation:

$$\begin{matrix} ( t ) = ( 1.0 - β ) [ \int {Q}_{p} d t + {Q}_{p} ] \\ = ( 1.0 - β ) [ {V}_{p} + {Q}_{p} ]\end{matrix}$$

Pₘᵤₛ is the amount of pressure provided by the patient's muscles. t is time in the continuous domain. Total pressure delivered to the patient is [ [symbol] ∫Qₚdt+Qₚ [symbol] ], which is the sum of the pressure contributions by the patient (Pₘᵤₛ) and the ventilator (Pᵥₑₙₜ or Target Pressure). β is the support setting (i.e., percentage of total support to be contributed by the ventilator). [symbol] is estimated patient resistance. [symbol] is estimated patient elastance. Qₚ is the flow rate into the patient. V_{P} is the volume going into the patient and is also represented as ∫Qₚdt. In alternative embodiments, the ventilator, controller, and/or effort module during the estimating operation 408 utilizes any suitable known system or method for calculating patient effort, such as ieSync, a physical sensor, and/or a muscle activity monitor.

Identification of respiratory system resistance and elastance is significant during the TAPA breath type. For example, both under and over estimates of resistance and elastance may significantly impair the synchrony between the patient and ventilator. Accordingly, in some embodiments, the ventilator and/or the effort module during the estimating operation 408 utilize a recursive least square adaptive algorithm to estimate resistance and elastance. The recursive least square adaptive algorithm guarantees that estimated resistance and compliance asymptomatically converge to real values in the patient's respiratory system. Therefore, the ventilator and/or the effort module utilizing a recursive least square adaptive algorithm during the estimating operation 408 accurately estimate resistance and compliance improving synchrony between the ventilator and patient when compared to ventilators that do not utilize the recursive least square adaptive algorithm to estimate resistance and compliance. In some embodiments, the recursive least square adaptive algorithm is illustrated below:

$${\hat{θ}}^{T} ( k ) = {\hat{θ}}^{T} ( k - 1 ) + F ( k ) ϕ ( k ) e° ( k )$$

$$e° ( k ) = ( \frac{{P}_{vent} ( k )}{β} + {P}_{vent} ( k ) ) - {ϕ}^{T} ( k ) \hat{θ} ( k - 1 )$$

$$F ( k ) = F ( k - 1 ) - \frac{F ( k - 1 ) ϕ ( k ) {ϕ}^{T} ( k ) F ( k - 1 )}{1 + {ϕ}^{T} ( k ) F ( k - 1 ) ϕ ( k )}$$

where θ^{T}(k)=[R_{P}(k) E_{P}(k)] is the patient respiratory parameters to be estimated;

$$ϕ ( k ) = [ \begin{matrix}{Q}_{p} ( k ) \\ {V}_{p } ( k )\end{matrix} ]$$

is the regression parameter vector, which can be directly measured or indirectly calculated; θ^{T}(k)=[ [symbol] (k) [symbol] (k)], which is the estimated patient respiratory parameter vector; and F(k)=F^{T}(k)>0 is the recursive least square gain at the computation cycle k.

Thus, an estimated resistance and elastance may be derived by the ventilator and/or the effort module during the estimating operation 408 using the recursive least square adaptive algorithm, as described above. Specifically, the parameter estimate vector update equation may solve for a recursive least squares gain value representing the resistance and elastance at a time instance based on a squared gain value for a previous time instance by subtracting a squared gain value for the previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the squared gain value for the previous time instance multiplied by the regression parameter vector at the time instance from a gain value for the previous time instance. The end result of the above calculation will provide an estimated resistance and elastance. In some embodiments, the recursive least square adaptive algorithm may be modified by introducing a forgetting factor 0<μ<1, such that the update equation becomes:

$$F ( k ) = \frac{1}{μ} [ F ( k - 1 ) - \frac{F ( k - 1 ) ϕ ( k ) {ϕ}^{T} ( k ) F ( k - 1 )}{μ + {ϕ}^{T} ( k ) F ( k - 1 ) ϕ ( k )} ]$$

In such instances, the closer μ is to 1, the less responsive the adaptive parameter estimation will be to parameter variations.

As illustrated, method 400 includes a calculating operation 410. During calculating operation 410, the ventilator, controller, and/or TAPA module, calculates a target inspiration pressure. During calculating operation 410, the ventilator, controller, and/or TAPA module, calculates a target inspiration pressure based at least on the time delay caused the control system, the estimated patient effort from the last computational cycle and the received support setting. Method 400 delivers ventilation according to the TAPA breath type, which is discussed in detail above. Accordingly, the ventilator, controller, and/or TAPA module, during the calculating operation 410, calculate a target inspiration pressure (Pᵥₑₙₜ) utilizing a target pressure equation that has been adjusted with a dynamic assist ratio. In some embodiments, the ventilator, controller, and/or TAPA module, during the calculating operation 410, calculate a target inspiration pressure (Pᵥₑₙₜ) utilizing the following adjusted effort equation:

$${P}_{vent} ( t ) = {\overline{G}}_{vent} ( s ) \cdot β \cdot ( \frac{ s +}{{R}_{P} s + {E}_{P}} ) ( {P}_{vent} ( t - \hat{τ} ) + ( t - \hat{τ} ) )$$

wherein Pᵥₑₙₜ is a target inspiration pressure, [symbol] is estimated patient effort, t is time in the continuous domain, β is a support setting, and \hat{τ} is an estimate of the control system delay. G̅ᵥₑₙₜ(s) is the transfer function representing dynamics of the control system with no delay The estimated/measured time delay \hat{τ} and the estimated/measured lung flow Q_{P}(t) and Q_{P}(t−\hat{τ}) are used to calculate the dynamic pressure assist ratio

$$β \cdot \frac{{Q}_{P} ( t )}{{Q}_{P} ( t - \hat{τ} )}$$

to deal with the control system delay and improve the patient-ventilator interaction.

Next, method 400 includes a delivering operation 412. During delivering operation 412, the ventilator and/or the pressure generating system deliver the target inspiration pressure to the patient in the next computational cycle. The delivering operation 412 is similar to the delivering operation 312 for method 300 described above.

As the ventilator performs the delivering operation 412, the ventilator performs the monitoring operation 406 again, as described above. Method 400 performs the monitoring operation 406, estimating operation 408, calculating operation 410, and delivering operation 412 repeatedly creating a closed-loop system of ventilation. In some embodiments, the ventilator during method 400 also performs the retrieving operation 404 repeatedly with the operations (406, 408, 410, and 412) listed above. In embodiments where the support setting is input or selected by the operator, the retrieving operation 404 will retrieve the same support setting until an operator inputs or selects a new support setting. In other embodiments where the support setting is determined by the ventilator based on patient parameters input or selected by the operator, the retrieving operation 404 will retrieve the same support setting until an operator inputs or selects a new patient parameters. Further, the ventilator, effort module, and/or controller during the estimating operation 408 estimates a new patient effort or updates the estimated patient effort after each computational cycle, such as the first, second, third, and etc. computational cycles during the TAPA breath type. The new or updated patient effort may be the same or different from the previous estimated patient efforts. Similarly, the ventilator, TAPA module, and/or controller during the calculating operation 410 calculates a new target inspiration pressure or updates the target inspiration pressure after each computational cycle, such as the delivery of a first, second, third, and etc. computational cycles during the TAPA breath type. The new or updated target inspiration pressure may be the same or different from the previously calculated target inspiration pressures.

In some embodiments, method 400 includes an initial delivering operation 402, which is similar to the initial delivering operation 302 described above. The ventilator and/or pressure generating system during the initial delivering operation 402 delivers an initial inspiration pressure to the patient during a first computational cycle.

In some embodiments, method 400 includes a displaying operation. The ventilator during the displaying operation displays any suitable information for display on a ventilator. In one embodiment, the displaying operation displays one or more of the breath type, the estimated patient effort, the calculated target pressure, the total pressure delivered, the monitored inspiration pressure, the monitored net lung volume, an initial inspiratory pressure, a list of delivered target inspiration pressures for a predetermined number of computational cycles, a list of estimated patient efforts from a predetermined number of computational cycles, a graph of the list of the delivered target inspiration pressure and/or the estimated patient efforts for a predetermined number of computational cycles, the support setting, a volume-assist setting, a flow-assist setting, and/or a time delay caused by the control system.

In some embodiments, a microprocessor-based ventilator that accesses a computer-readable medium, which can be transitory or non-transitory, having computer-executable instructions for performing the method of ventilating a patient with a medical ventilator is disclosed. This method includes repeatedly performing all or a portion of the steps disclosed in methods 200, 300, and 400 as described above and as illustrated in FIGS. 2, 3, and 4 with the modules as described above and/or as illustrated in FIG. 1.

In some embodiments, the ventilator system includes means for performing all or a portion of the steps disclosed in methods 200, 300, and 400 as described above and as illustrated in FIGS. 2, 3, and/or 4. The means for performing these embodiments are illustrated in FIG. 1 and described above.

### EXAMPLES

The examples listed below are exemplary only and not meant to be limiting of the disclosure.

### Example 1

The Nyquist method was employed to compare the closed-loop stability margin between the NPA breath type and the conventional PA breath type. For a closed-loop control system, the Nyquist plot of its open loop response G₀(jω) shows the information of phase margin, gain margin, and the maximum sensitivity magnitude, i.e. |s(jω)|ₘₐₓ where s(jω) represents the sensitivity function of the closed-loop system.
1. On Nyquist curve of G₀(jω), the maximum value |s(jω)|ₘₐₓ is the inverse of the minimum value of the distance between the G₀(jω) curve and the point (−1,0), i.e.

$$\frac{1}{{\vert 1 + {G}_{o} ( jω ) \vert}_{min}} .$$

The minimum value |1+G₀(jω)|ₘᵢₙ represents the stability margin of the closed-loop system. The larger this minimum value, the larger the stability margin.
2. The gain margin is defined as:

$$GM = \frac{1}{\vert {G}_{o} ( - jπ ) \vert} .$$

The larger the GM, the better stability.
   Two sets of data as shown in Table 1 are used for stability margin comparison. The first set of data shows under-estimates of respiratory parameters, i.e. [symbol] <R_{P} and [symbol] <Eᵣ; while the second set shows over-estimates of respiratory parameters, i.e., [symbol] >R_{P} and [symbol] >E_{P}. In both cases, K_{f}=K_{V}=0.8 and the corresponding β=4.0.

TABLE 1
Under-estimate and over-estimate parameters for simulation.

| Respiratory Parameter | Case 1 (under-estimate) | Case 2 (over-estimate) |
|---|---|---|
| R_{P} | 10.0 | 10.0 |
| \hat{R_{P}} | 9.0 | 12.0 |
| E_{P} | 0.05 | 0.05 |
| \hat{E_{P}} | 0.045 | 0.055 |
| K_{f} | 0.8 | 0.8 |
| K_{V} | 0.8 | 0.8 |
| β | 4 | 4.0 |

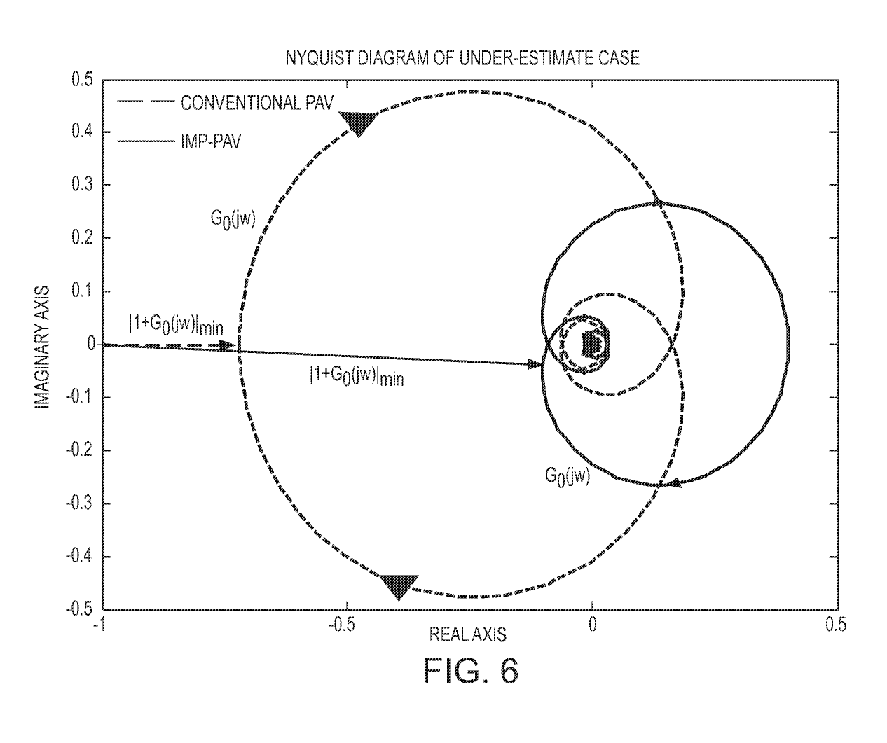

FIG. 6 illustrate a stability margin comparison of a NPA breath type and a PA breath type using Nyquist plots and the simulation respiratory parameters listed under case 1 from Table 1 for under-estimates. Based on this comparison, FIG. 6 illustrates that the minimum distance between the G₀(jω) and the (−1,0) line for the NPA breath type is larger than the line for the PA breath type. Moreover, the gain margin in NPA breath type is also larger than the gain margin for the PA breath type. Accordingly, the closed-loop system for the NPA breath type has a larger stability margin than the closed-loop system for the PA breath type for case 1.

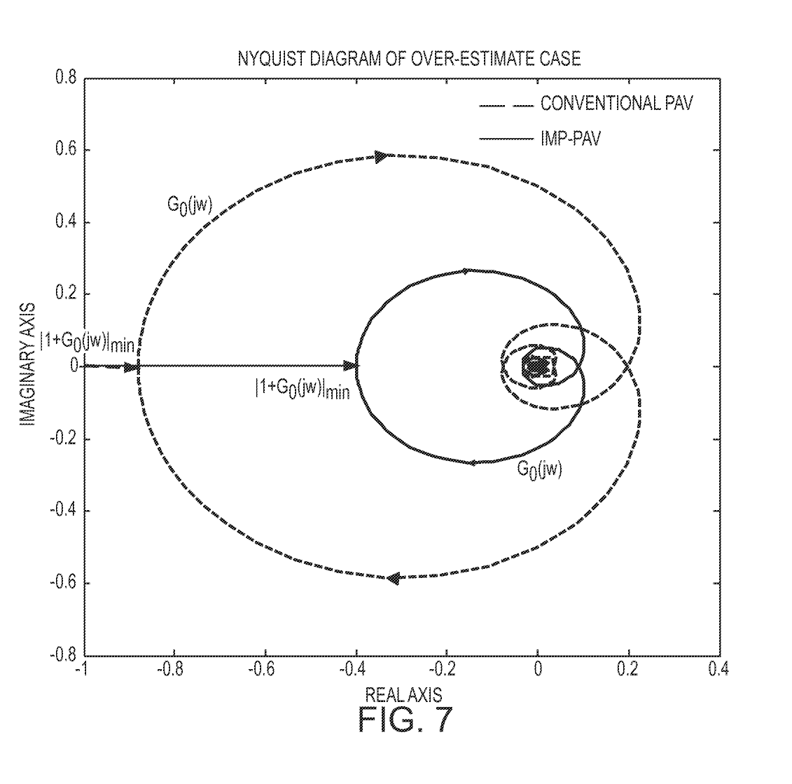

FIG. 7 illustrate a stability margin comparison of a NPA breath type and a PA breath type using Nyquist plots and the simulation respiratory parameters listed under case 2 from Table 1 for over estimates. FIG. 7 shows that the minimum distance between the G₀(jω) and the (−1,0) line for the NPA breath type is larger than the line for the PA breath type. Moreover, the gain margin in NPA breath type is also larger than the gain margin for the PA breath type. Accordingly, the closed-loop system for the NPA breath type has a larger stability margin than the closed-loop system for the PA breath type for case 2.

Those skilled in the art will recognize that the methods and systems of the present disclosure may be implemented in many manners and as such are not to be limited by the foregoing exemplary embodiments and examples. In other words, functional elements being performed by a single or multiple components or modules, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components or modules, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces of modules and other components, and those variations and modifications that may be made to the hardware or software firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A method for ventilating a patient with a ventilator comprising:
delivering an initial inspiration pressure to a patient in a first computational cycle;
retrieving a support setting;
monitoring inspiration flow during the first computation cycle;
estimating a first patient effort utilizing an inverse model based at least on the inspiration flow monitored during the first computational cycle;
calculating a first target inspiration pressure based at least on the first estimated patient effort from the first computational cycle and the support setting; and
delivering the first target inspiration pressure to the patient in a second computational cycle.

2. The method of claim 1, further comprising:
monitoring the inspiration flow during the second computational cycle;
estimating a second patient effort utilizing the inverse model based at least on the inspiration flow monitored during the second computational cycle;
calculating a second target inspiration pressure based at least on the second estimated patient effort and the support setting; and
delivering the second target inspiration pressure to the patient in a third computational cycle,
wherein the steps of the method form a closed-loop system that is a negative feedback system.

3. The method of claim 2, wherein the step of estimating first patient effort utilizing the inverse model and the step of estimating the second patient effort utilizing the inverse model are performed utilizing a following patient effort equation:
$$( t ) = {Q}_{P} \frac{+ s}{s} - {P}_{vent}$$
wherein the step of calculating the first target inspiration pressure and the step of calculating the second inspiration target pressure are performed by utilizing a following target pressure equation:
Pᵥₑₙₜ(t)=β· [symbol] (t)
wherein Pᵥₑₙₜ is a target inspiration pressure, [symbol] is an estimated patient effort, t is time in the continuous domain, β is the support setting, [symbol] is estimated patient resistance, [symbol] is estimated patient elastance, s denotes a complex variable in an s-domain, and Qₚ is the flow rate into the patient.

4. The method of claim 3, wherein the patient elastance and the patient resistance are estimated utilizing a recursive least square adaptive algorithm.

5. The method of claim 4, wherein the step of calculating the first target inspiration pressure and the step of calculating the second target inspiration pressure are adjusted to remove any time delay caused by a control system of the ventilator.

6. The method of claim 2, wherein the step of estimating the first patient effort utilizing the inverse model and the step of estimating the second patient effort utilizing the inverse model are performed utilizing a following patient effort equation:
$$( t ) = {Q}_{P} \frac{ s +}{s} - {P}_{vent}$$
wherein the step of calculating the first target inspiration pressure and the step of calculating the second inspiration target pressure are adjusted utilizing a dynamic assist ratio and are performed with a following equation:
$${P}_{vent} ( t ) = {\overline{G}}_{vent} ( s ) \cdot {e}^{- \hat{τ} s} \cdot β \cdot \frac{ ( t )}{ ( t ) ( t - \hat{τ} )} ( t )$$
wherein Pᵥₑₙₜ is a target inspiration pressure, [symbol] is an estimated patient effort, t is time in the continuous domain, β is the support setting,
$$β \cdot \frac{ ( t )}{ ( t ) ( t - \hat{τ} )}$$
is the dynamic assist ratio, [symbol] is estimated patient resistance, [symbol] is estimated patient elastance, s denotes a complex variable in an s-domain, G̅ᵥₑₙₜ(s) is a transfer function representing dynamics of a control system with no delay, e is an exponential function, and \hat{τ} is an estimate of a control system delay

7. The method of claim 1, wherein the step of calculating the first target inspiration pressure is adjusted to remove any time delay caused by a control system of the ventilator.

8. The method of claim 7, wherein the step of calculating the first target inspiration pressure is adjusted utilizing a dynamic assist ratio with a following equation:
$${P}_{vent} ( t ) = {\overline{G}}_{vent} ( s ) \cdot {e}^{- \hat{τ} s} \cdot β \cdot \frac{ ( t )}{ ( t ) ( t - \hat{τ} )} ( t )$$
wherein Pᵥₑₙₜ is a target inspiration pressure, [symbol] is an estimated patient effort, t is time in the continuous domain, β is the support setting,
$$β \cdot \frac{ ( t )}{ ( t ) ( t - \hat{τ} )}$$
is the dynamic assist ratio, G̅ᵥₑₙₜ(s) is a transfer function representing dynamics of the control system with no delay, s denotes a complex variable in an s-domain, e is an exponential function, and \hat{τ} is an estimate of a control system delay.

9. A method for ventilating a patient with a ventilator comprising:
delivering an initial inspiration pressure to a patient in a first computational cycle;
retrieving a support setting;
monitoring inspiration flow during the first computational cycle;
estimating a first patient effort utilizing at least the inspiration flow monitored during the first computational cycle;
calculating a first target inspiration pressure based at least on the first estimated patient effort from the first computational cycle, the support setting, and a time delay caused by a control system of the ventilator; and
delivering the first target inspiration pressure to the patient in a second computational cycle.

10. The method of claim 10, further comprising:
monitoring the inspiration flow during the second computational cycle;
estimating a second patient effort utilizing at least the inspiration flow monitored during the second computational cycle;
calculating a second target inspiration pressure based at least on the second estimated patient effort from the second computational cycle, the support setting, and the time delay caused by the control system of the ventilator; and
delivering the second target inspiration pressure to the patient in a third computational cycle.

11. The method of claim 10, wherein the step of calculating the first target inspiration pressure and the step of calculating the second inspiratory target pressure are adjusted for the time delay by utilizing a dynamic assist ratio with a following equation:
$${P}_{vent} ( t ) = {\overline{G}}_{vent} ( s ) \cdot β \cdot ( \frac{\hat{{R}_{p}} s + \hat{{E}_{p}}}{{R}_{p} s + {E}_{p}} ) ( {P}_{vent} ( t - \hat{τ} ) + ( t - \hat{τ} )$$
wherein Pᵥₑₙₜ is a target inspiration pressure, [symbol] is an estimated patient effort, t is time in the continuous domain, β is the support setting,
$$β \cdot \frac{{Q}_{p} ( t )}{{Q}_{p} ( t - \hat{τ} )}$$
is the dynamic assist ratio, G̅ᵥₑₙₜ(s) is a transfer function representing dynamics of the control system with no delay, [symbol] is estimated patient resistance, [symbol] is estimated patient elastance, R_{P} is patient resistance, E_{P} is patient elastance, s denotes a complex variable in an s-domain, and \hat{τ} is an estimate of a control system delay.

12. The method of claim 11, wherein the patient elastance and the patient resistance are estimated utilizing a recursive least square adaptive algorithm.

13. A ventilator system comprising:
a pressure generating system that generates a flow of breathing gas;
a ventilation tubing system including a patient interface for connecting the pressure generating system to a patient;
one or more sensors operatively coupled to at least one of the pressure generating system, the patient, and the ventilation tubing system, wherein the one or more sensors generate output indicative of at least an inspiration flow;
an inverse model (IM) effort module that calculates an estimated patient effort for each computational cycle utilizing an inverse model based on the output indicative of at least the inspiration flow from a last computational cycle; and
a negative proportional assist (NPA) module that receives a support setting, receives an estimated patient effort from the IM effort module for each computational cycle, calculates a target inspiration pressure based at least on the received support setting and the estimated patient effort received from the IM effort module for the last computational cycle, and sends instructions to the pressure generating system to deliver the calculated target inspiration pressure in a next computational cycle to the patient during a negative proportional assist (NPA) breath type,
wherein the instructions sent by the IM effort module and the NPA module provide closed-loop ventilation that is a negative feedback system.

14. The ventilator system of claim 13, wherein the IM effort module calculates an estimated patient effort utilizing the inverse model by utilizing a following patient effort equation:
$$( t ) = {Q}_{P} \frac{\hat{{R}_{p}} s + \hat{{E}_{p}}}{s} - {P}_{vent}$$
wherein the NPA module calculates the target inspiration pressure by utilizing a following target pressure equation:
Pᵥₑₙₜ(t)=β· [symbol] (t)
wherein Pᵥₑₙₜ is a target inspiration pressure, [symbol] is an estimated patient effort, t is time in the continuous domain, β is the support setting, [symbol] is estimated patient resistance, [symbol] is estimated patient elastance, s denotes a complex variable in an s-domain, and Qₚ is the flow rate into the patient.

15. The ventilator system of claim 14, wherein the patient elastance and the patient resistance are estimated utilizing a recursive least square adaptive algorithm.

16. The ventilator system of claim 15, wherein the NPA module adjusts the target inspiration pressure to remove any time delay caused by a control system of the ventilator system.

17. The ventilator system of claim 16, wherein the NPA module adjusts the target inspiration pressure with a dynamic assist ratio by utilizing a following equation instead of the patient effort equation listed above:
$${P}_{vent} ( t ) = {\overline{G}}_{vent} ( s ) \cdot {e}^{- \hat{τ} s} \cdot β \cdot \frac{ ( t )}{ ( t ) ( t - \hat{τ} )} ( t )$$
wherein G̅ᵥₑₙₜ(s) is a transfer function representing dynamics of the control system with no delay,
$$β \cdot \frac{ ( t )}{ ( t ) ( t - \hat{τ} )}$$
is the dynamic assist ratio, e is an exponential function, and \hat{τ} is an estimate of a control system delay.

18. The ventilator system of claim 13, wherein the NPA module adjusts the target inspiration pressure to remove any time delay caused by a control system of the ventilator system.

19. The ventilator system of claim 19, wherein the NPA module adjusts the target inspiration pressure utilizing a dynamic assist ratio with a following equation:
$${P}_{vent} ( t ) = {\overline{G}}_{vent} ( s ) \cdot {e}^{- \hat{τ} s} \cdot β \cdot \frac{ ( t )}{ ( t ) ( t - \hat{τ} )} ( t )$$
wherein Pᵥₑₙₜ is a target inspiration pressure, [symbol] is an estimated patient effort, t is time in the continuous domain, β is the support setting,
$$β \cdot \frac{ ( t )}{ ( t ) ( t - \hat{τ} )}$$
is the dynamic assist ratio, G̅ᵥₑₙₜ(s) is a transfer function representing dynamics of the control system with no delay, s denotes a complex variable in an s-domain, e is an exponential function, and \hat{τ} is an estimate of a control system delay.

20. The ventilator system of claim 13, further comprising a trigger module that delivers a breath to the patient based on the output indicative of at least the inspiration flow.
